(19) United States
(12) Patent Application Publication
(10) Pub. No.: US 2023/0172982 A1
(43) Pub. Date: Jun. 8, 2023

(54) Elimination of BCMA-positive malignancies by CAR expressing NK cells

(71) Applicant: ImmunityBio, Inc., Culver City, CA (US)

(72) Inventors: Laurent H. Boissel, Culver City, CA (US); Hans G. Klingemann, Culver City, CA (US); Barry J. Simon, M.D., Culver City, CA (US); Abhijit Dandapat, Culver City, CA (US)

(21) Appl. No.: 17/795,735

(22) PCT Filed: Jan. 30, 2020
(86) PCT No.: PCT/US2020/015897

§ 371 Date: Jul. 27, 2022

(51) Int. Cl. A61K 35/17; C07K 14/495; C07K 14/54; C07K 14/735; C07K 14/715; C07K 14/725; C07K 14/55

(52) U.S. Cl. CPC A61K 35/17; C07K 14/495; C07K 14/5434; C07K 14/70535; C07K 14/7151; C07K 14/7051; C07K 14/55; C07K 14/5443; C07K 2317/622; C07K 2317/53

(57) ABSTRACT

Provided are genetically modified NK cells expressing a chimeric antigen receptor targeting an BCMA superfamily receptor. The CAR can comprise an intracellular domain of FcεRIγ and further recombinant proteins expressed by the genetically modified NK cells are CD16, autocrine growth stimulating cytokines, and optionally one of IL-12, a TGF-beta trap, or a homing receptor. Also described are methods for treating a patient having or suspected of having a disease that is treatable with NK-92 cells, such as cancer, comprising administering to the patient the genetically modified NK cells.

### SEQUENCE LISTING

The content of the XML text file of the sequence listing named Seq_listing_20200127_0019US, which is 69.7 KB in size was created on Jul. 12, 2022 and electronically submitted via EFS-Web along with the application is incorporated by reference in its entirety.

### FIELD OF THE INVENTION

The field of the invention is genetically modified immune competent cells that express a chimeric antigen receptor (CAR), and particularly modified NK-92 cells expressing a CAR with an Fc epsilon receptor gamma (FcεRIγ) signaling domain targeting a B-Cell Maturation Antigen (BCMA) receptor.

### BACKGROUND OF THE INVENTION

B-Cell Maturation Antigen (BCMA) is membrane receptor that is exclusively expressed in plasmablasts and differentiated plasma cells, where it is required for cell survival, but is not expressed in earlier stages B-cells, hematopoietic stem cells, or other normal tissue cells. Surface expression of BCMA is increased in multiple myeloma plasmacytoid B cells and has also been detected in a subtype of Hodgkin's disease. The very narrow expression profile of BCMA and its absence from normal tissues make it a particularly promising target for therapy, and multiple therapeutic approaches have been and are being developed, such as antibody-drug conjugates (ADC), bispecific T-cell engager (BiTE), CAR-T cells (CAR-T), bispecific molecules, and bi/tri specific antibodies, as well as cancer vaccines. However, ADC and bi/trispecific antibodies may suffer from poor tissue penetration. BiTE have short serum half-life and have also been shown to induce CRS and neurotoxicity. CAR-T cell therapy can be associated with significant adverse events (CRS and related neurotoxicity). Because of the risk of GvHD associated with allogeneic T-cells infusion, current CAR-T technology often relies on engineering autologous T-cells, which results in patient-to-patient variability, as well as exclusion of patients whose T-cells cannot be expanded.

Natural killer (NK) cells are cytotoxic lymphocytes that constitute a significant component of the innate immune system. In most cases, NK cells represent about 10-15% of circulating lymphocytes, and bind and kill targeted cells, including virus-infected cells and many malignant cells. NK cell killing is non-specific regarding particular antigens and can occur without prior immune sensitization. Killing of targeted cells is typically mediated by cytolytic proteins, including perforin, granzymes, and granulysin.

Autologous NK cells have been used as therapeutic entities. To that end, NK cells are isolated from the peripheral blood lymphocyte fraction of whole blood, expanded in cell culture to obtain sufficient numbers of cells, and then re-infused into a subject. Autologous NK cells have shown in at least some cases moderate effectiveness in both ex vivo therapy and in vivo treatment. However, isolation and growth of autologous NK cell is time and cost intensive. Moreover, autologous NK cell therapy is further limited by the fact that not all NK cells are cytolytic.

At least some of these difficulties can be overcome by use of NK-92 cells, which are a cytolytic cancer cell line which was discovered in the blood of a subject suffering from a non-Hodgkins lymphoma and then immortalized in vitro (Gong et al., Leukemia 8:652-658 (1994)). While NK-92 cells are NK cell derivatives, NK-92 cells lack the major of inhibitory receptors that are otherwise displayed by normal NK cells, and retain most of the activating receptors. NK-92 cells do not, however, attack normal cells nor do they elicit an unacceptable immune rejection response in humans. Due to these desirable characteristics, NK-92 cells were characterized in detail and explored as therapeutic agent in the treatment of certain cancers as is described, for example, in WO 1998/049268 or US 2002/068044.

Phenotypic changes distinguishing a tumor cell from normal cells derived from the same tissue are often associated with one or more changes in the expression of specific gene products, including the loss of normal cell surface components or the gain of others (i.e., antigens not detectable in corresponding normal, non-cancerous tissue). The antigens which are expressed in neoplastic or tumor cells, but not in normal cells, or which are expressed in neoplastic cells at levels substantially above those found in normal cells, have been termed “tumor-specific antigens” or “tumor-associated antigens.” Such tumor-specific antigens may serve as markers for tumor phenotype. Tumor-specific antigens include cancer/testis-specific antigen (e.g. MAGE, BAGE, GAGE, PRAME and NY-ESO-1), melanocyte differentiation antigens (e.g. tyrosinase, Melan-A/MART, gp100, TRP-1 and TRP-2), mutated or aberrantly expressed antigens (e.g. MUM-1, CDK4, beta-catenin, gp100-in4, p15 and N-acetylglucos-aminyltransferase V), and antigens that are expressed at higher levels in tumors (e.g., CD19 and CD20).

Tumor-specific antigens have been used as targets for cancer immunotherapies. One such therapy utilizes chimeric antigen receptors (CARs) expressed on the surface of immune cells, including T cells and NK cells, to improve cytotoxicity against cancer cells. CARs comprise a single-chain variable fragment (scFv) linked to at least one intracellular signaling domain. The scFv recognizes and binds an antigen on the target cell (e.g., a cancer cell) and triggers effector cell activation. The signaling domains contain immunoreceptor tyrosine-based activation domains (ITAMs) that are important for intracellular signaling by the receptor.

The first generation of CARs used in T-cells contained one cytoplasmic signaling domain. For example, one version of a first-generation CAR in T-cells included a signaling domain from the Fc epsilon receptor gamma (FcεRIγ) which contained one ITAM, while another version contained the signaling domain from CD3ζ which contained three ITAMs. In vivo and in vitro studies showed that the CD3ζ CAR T-cells were more efficient at tumor eradication than FcεRIγ CAR T-cells (e.g., Haynes, et al. 2001, 1 J. Immunology 166:182-187; Cartellieri, et al. 2010, J. Biomed and Biotech, Vol. 2010, Article ID 956304). Additional studies then revealed that certain costimulatory signals were required for full activation and proliferation of such recombinant T-cells, and second and third generation CARs combined multiple signaling domains into a single CAR to enhance efficacy of the recombinant CAR T-cells. Due to their less desirable philological effects in the tested T-cells, first generation CARs and the FcεRIγ signaling domains were largely discarded in favor of the new, more efficient CARs using CD3ζ in combination with one or more additional signaling domains (e.g., Hermanson and Kaufman 2015, Frontiers in Immunol., Vol. 6, Article 195).

More recently, selected CARs have also been expressed in NK cells. For example, CAR-modified NK-92 cells have used first generation CARs with only a CD3ζ intracellular signaling domain. Several antigens have been targeted by these first generation CAR-NK cells, including CD19 and CD20 for B cell lymphoma, ErbB2 for breast, ovarian, and squamous cell carcinoma, GD2 for neuroblastoma, and CD138 for multiple myeloma. Second generation CAR-NK cells from the NK-92 line have also been created for several antigens, including EpCAM for multiple carcinomas HLA-A2 EBNA3 complex for Epstein—Barr virus, CS1 for multiple myeloma, and ErbB2 for HER2 positive epithelial cancers. The most common intracellular costimulatory domain used alongside CD3ζ in second generation NK-92 CARs is CD28. However, the potential effect of the CD28 domain is unclear since NK cells do not naturally express CD28. Additional second generation CARs have incorporated the 4-1BB intracellular signaling domain along with CD3ζ to improve NK cell persistence. Others compared functionality of different intracellular domains using an ErbB2 scFv fused with CD3ζ alone, CD28 and CD3ζ, or 4-1BB and CD3ζ tested against breast cancer cells. They found that both of the second generation constructs improved killing compared to the first generation CARs and the CD28 and CD3ζ had 65% target lysis, the 4-1BB and CD3ζ lysed 62%, and CD3 alone killed 51% of targets. 4-1BB and CD28 intracellular domains were also compared in a recent study using anti-CD19 CARs expressed on NK-92 cells for B cell malignances. Still others found that CD3ζ/4-1BB constructs were less effective than CD3ζ/CD28 in cell killing and cytokine production, highlighting differential effects of CD28 and 4-1BB costimulatory domains.

Third generation NK-92 CARs were constructed of an anti-CD5 scFv with CD3ζ, CD28, and 4-1BB intracellular signaling domains and demonstrated specific and potent anti-tumor activity against a variety of T-cell leukemia and lymphoma cell lines and primary tumor cells. Such cells were also able to inhibit disease progression in xenograft mouse models of T cell Acute lymphoblastic leukemia (ALL) cell lines as well as primary tumor cells (Transl Res. 2017 September ; 187: 32-43). In further examples, WO 2016/201304 and WO 2018/076391 teach use of third generation CD3ζ CARs expressed in NK cells and NK-92 cells.

However, NK cells (and particularly NK-92 cells) are often difficult to genetically modify as evidenced by numerous failures to engineer NK-92 cells to express an Fc receptor. Such difficulties are further compounded where NK-92 cells are transfected with multiple recombinant genes or relatively large recombinant nucleic acid payload for heterologous expression. Additionally, NK-92 cells also exhibit a significant lack of predictability with respect to recombinant expression of exogenous proteins (e.g., CD16). On a functional level, while exhibiting in most cases targeted cytotoxicity, most if not all CAR NK-92 cells require a high effector to target cell ratio.

In addition, even where cytotoxic cells expressing a CAR are relatively effective in vitro, various suppressive or inhibitory factors associated with the tumor microenvironment in vivo may reduce or even abrogate cytotoxicity of such cells. Similarly, and despite the expression of a CAR, such modified cells may not always be effective in targeting the tumor microenvironment.

Therefore, even though numerous recombinant NK-92 cells are known in the art, all or almost all of them suffer from various difficulties. Furthermore, while therapeutic approaches targeting BCMA are being developed, they have various disadvantages as well. Consequently, there remains a need for CAR-expressing NK-92 cells that express a high-activity CAR in significant quantities and that targets BCMA, that can be readily cultivated in a simple and effective manner, and that have high cytotoxicity in a tumor microenvironment to eliminate BCMA positive malignancies.

### SUMMARY OF THE INVENTION

The inventors have discovered that natural killer (NK) cells, and particularly NK-92 cells, may be genetically modified to express a target-specific CAR and further recombinant proteins to increase CAR mediated cell killing, ADCC, and cytotoxicity in and/or homing to a tumor microenvironment. In addition, such recombinant cells also expressed cytokines for autocrine growth stimulation that advantageously assists in clonal selection of modified cells.

In one aspect of the inventive subject matter, the inventors contemplate a genetically modified NK cell (and especially an NK-92 cell) that expresses (i) a membrane bound recombinant chimeric antigen receptor (CAR) that comprises in a single polypeptide chain an extracellular binding domain, a hinge domain, a transmembrane domain, and a signaling domain, wherein the extracellular binding domain specifically binds to a BCMA receptor; (ii) a recombinant CD16; (iii) an autocrine growth stimulating cytokine; and (iv) optionally one of IL-12, a TGF-beta trap, or a homing receptor.

In some embodiments, the extracellular binding domain comprises a scFv, and/or the signaling domain comprises a FcεRIγ signaling domain. In further contemplated aspects, the recombinant CD16 is a CD16^{158V} mutant, and/or the autocrine growth stimulating cytokine is IL-2 or IL-15, which may additionally comprise an endoplasmic retention sequence. In still further contemplated embodiments, the IL-12 is a single chain IL-12 heterodimer, and the TGF-beta trap comprises a single chain dimer of the TGF-beta Receptor II ectodomain and is preferably secreted. Preferred homing receptors include cell adhesion molecules, selectins, integrins, a C—C chemokine receptor, or a C—X—C chemokine receptor. For example, suitable homing receptor include CCR1, CCR2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1, CCXCKR, D6, DARC, and the receptor for CXCL14.

Viewed from a different perspective, genetically modified NK cells are contemplated that include a nucleic acid that encodes a (i) membrane bound recombinant chimeric antigen receptor (CAR) that comprises in a single polypeptide chain an extracellular binding domain, a hinge domain, a transmembrane domain, and a signaling domain, wherein the extracellular binding domain specifically binds to a BCMA receptor; (ii) a recombinant CD16; (iii) an autocrine growth stimulating cytokine; and (iv) optionally one of IL-12, a TGF-beta trap, or a homing receptor. With regard to the NK cell and the expressed proteins, the same considerations as noted above apply.

In another aspect of the inventive subject matter, the inventors also contemplate a method of treating cancer in a patient in need thereof in which the patient receives a therapeutically effective amount of the genetically modified NK cells presented herein, thereby treating the cancer. Where desired, contemplated methods may also include a step of administering at least one additional therapeutic entity selected from the group consisting of a viral cancer vaccine, a bacterial cancer vaccine, a yeast cancer vaccine, N-803, an antibody, a stem cell transplant, and a tumor targeted cytokine. For example, contemplated cancers include lung cancer, a breast cancer, a thyroid cancer, an esophageal cancer, a gastric cancer, a gastroesophageal cancer, or a head and neck cancer. Most typically, about 1×10⁸ to about 1×10¹¹ cells per m² of body surface area of the patient are administered to the patient. Therefore, the inventors also contemplate use of a genetically modified NK cell in the treatment of cancer.

Various objects, features, aspects and advantages of the inventive subject matter will become more apparent from the following detailed description of preferred embodiments along with the accompanying drawing figures in which like numerals represent like components.

### BRIEF DESCRIPTION OF THE DRAWINGS

FIG. 1 is a schematic representation of exemplary CAR constructs. All CAR variants had an extracellular domain comprising an anti-BCMA receptor scFv region, a hinge region from CD8 (CD8 hinge), a transmembrane domain from CD28 (CD28 TM), and an intracellular domain from FcεRIγ.

FIG. 2 is a schematic representation of a background of BCMA in normal physical functions in the plasma cells (PC), as well as in the PC of multiple myeloma (MM) patients.

FIG. 3 illustrates one embodiment of a schematic representation of the cloning of BCMA t-haNK constructs as illustrated herein. The BCMA VH and VL fragments may have sequences depicted in SEQ ID Nos:39-42.

FIG. 4 depicts exemplary flow cytometry analysis results of BCMA.CAR and CD16 proteins expression on aNK, haNK, and BCMA t-haNK cells is illustrated in the top panel. The bottom panel illustrates CAR and CD16 proteins expression on BCMA t-haNK cell constructs having VH-VL scFv CAR and VL-VH scFv CAR respectively.

FIG. 5 depicts exemplary CAR cytotoxicity of BCMA t-haNK polyclonal cell populations. The left panel illustrates BCMA antigen specific cytotoxicity on SUP-B15 BCMA+, while the right panel illustrates spontaneous cytotoxicity on K562.

FIG. 6 depicts exemplary ADCC assay of BCMA t-haNK polyclonal cell populations.

FIG. 7 depicts exemplary flow cytometry analysis results on BCMA t-haNK clones.

FIG. 8 depicts exemplary BCMA CAR and CD16 expression analysis of BCMA t-haNK clones using flow cytometry. Clones 1-4 were from VH-VL pool # 2, while clones 5-13 were from VH-VL pool # 3.

FIG. 9 depicts another exemplary flow cytometry analysis results on BCMA t-haNK clones.

FIG. 10 depicts exemplary BCMA CAR and CD16 expression analysis of BCMA t-haNK clones using flow cytometry. Clone 15 was from VL-VH pool # 1, clones 21-25 were from VL-VH pool # 3, and clones 27-47 were from VH-VL pool # 1.

FIG. 11 depicts exemplary CAR and natural cytotoxicity of BCMA tri-cistronic clones. The left panel illustrates natural cytotoxicity of BCMA t-haNK cells against K562 cell line. The right panel illustrates CAR mediated cytotoxicity of BCMA t-haNK cells against SUP-B15 BCMA+cell line.

FIG. 12 depicts exemplary ADCC activity of BCMA tri-cistronic selected clones against the SUPB-15 BCMA-/CD20+cell line.

FIG. 13 depicts exemplary sequence verification of TGF-β trap/BCMA Quadracistronic (quad) DNA clone # 11.

FIG. 14 depicts exemplary expression analysis of BCMA TGF-β trap/BCMA Quadracistronic (quad) t-haNK polyclonal cell population.

FIG. 15 depicts exemplary CAR and natural cytotoxicity of TGF-β trap/BCMA quadracistronic t-haNK cells. The left panel illustrates CAR mediated cytotoxicity of TGF-β trap/BCMA Quad on SUP-B15 BCMA+cell line. The right panel illustrates natural cytotoxicity of TGF-β trap/BCMA Quad on K562 cell line.

FIG. 16 depicts exemplary ADCC of TGF-β trap/BCMA Quadracistronic (quad) t-haNK polyclonal cell population on SUP B15-19KO/CD20 cell line.

### DETAILED DESCRIPTION OF THE INVENTION

The inventors have discovered that genetically modified NK cells can have significant general toxicity, target specific CAR-mediated cytotoxicity, and ADCC (antibody dependent cellular cytotoxicity), and that such genetically modified cells can be grown under autocrine growth stimulation that will also confer selective effect towards successfully transfected cells. In addition, contemplated cells may further express from a recombinant nucleic acid secreted IL-12 and/or a TGF-beta trap to reduce or eliminate an immune-suppressive environment. Additionally, or alternatively, contemplated cells may express from a recombinant nucleic acid a homing receptor. Therefore, modified cells may be generated by transfection with a tricistronic or quadracistronic nucleic acid.

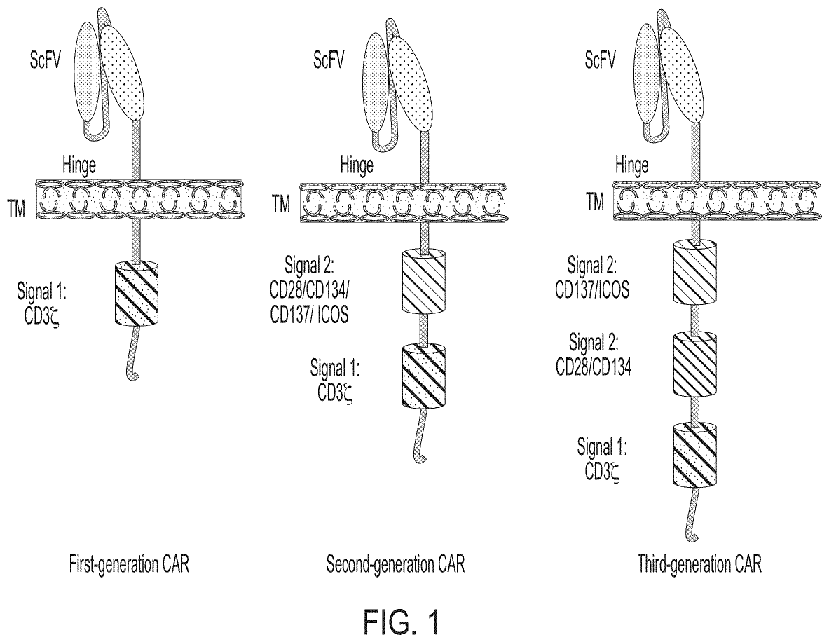

CAR combine an extracellular antigen-recognizing part, usually derived from the variable domain of a specific antibody, to an intracellular signaling domain, which may be either single or with additional co-stimulatory elements. Such a CAR that can trigger a cytolytic response once a specific antigen is recognized. Illustrative examples of CARs are provided in FIG. 1.

The inventors have now developed CAR structures that specifically recognize BCMA. In preferred embodiments, the CAR is a first-generation CAR, as illustrated in FIG. 1. This CAR enables effector cells to specifically engage and kill BCMA-positive target cells. This DNA sequence encoding the BCMA is typically integrated into a bicistronic CD16 (158V)-erIL-2 plasmid in a way that allows simultaneous expression of three elements: (a) BCMA.CAR, which allows for specific killing of BCMA-positive cells; (b) CD16 (158V), which enables ADCC when combined with a therapeutic monoclonal antibody; and (c) erIL2, which allows cell expansion in absence of exogenous IL-2 and maintains selective pressure for transgene expression. The CAR and CD16 (158V) are preferably separated by a 2A sequence that allows equimolar expression of 2 proteins from a single mRNA. CD16(158V) and ERIL2 are separated by an IRES sequence that allows internal translation initiation.

The tricistronic plasmid designed as described above is transfected into aNK cells, using electroporation. Stable expression of the tricistronic transgene is ensured by growth in IL-2 free conditions. Expression of CAR and CD16(158V) may be measured as described in more detail below. The inventors also showed that a so generated cell population was able to grow in absence of IL-2, was >80% pure, and expressed a high level of both CAR and CD16.

Specific targeting of BCMA-positive malignancies is currently achieved by antibody-drug conjugates (ADC), bispecific T-cell engager (BiTE), CAR T cell (CAR T), bispecific molecule, and bi/trispecific antibodies. Antibody-based molecules (ADC, bi/trispecific Abs) are very specific but are not able to access all parts of the body, particularly the brain. BiTEs have better diffusion properties than Abs but display very short serum half-like, which may require them to be infused continuously for a prolonged activity. They also have been shown to be associated with significant risks of CRS and neurotoxicity. Moreover, they functionally depend on the patient's cytotoxic cells (T- and NK cells), which may display highly impaired activity depending on patient treatment history, as well as patient to patient variability. The cell therapy products and methods disclosed herein overcome the disadvantages above since the product and methods are targeted exclusively to BCMA, allow for passage across the brain-blood barrier, and is not dependent on the patient's cells.

BCMA-targeted CAR-T cells have been designed but currently rely on engineering and expanding the patient's own T-cells (autologous). This can lead to variability in yields of expansion between patients, as well as in T-cell subsets composition. The disclosure herein overcomes these defects because cell therapy products as presented herein include clonal cell lines with no limit of expansion and no batch-to-batch variability, and therefore is available to all patients.

Allogeneic CAR-T therapy using engineered T-cells from healthy donors requires further cellular engineering to remove the risks of causing Graft versus host disease (GvHD). The parental cell line disclosed herein does not cause GvHD. CAR-T infusions often result in severe adverse events such as CRS and neurotoxicity, which can be treated with appropriate care but can still be fatal. Infusions of the parental cell line to the invention (aNK) did not cause any severe adverse events. Successful responses with CAR-T therapy are usually accompanied by expansion and long-term persistence of the CAR-T cells, which can cause long-term toxicities due to on-target/off-tumor activity. The cell therapy products disclosed herein does not engraft and is not long-lived, and therefore cannot cause long-lasting toxicities.

It should be further appreciated that FcεRIγ-containing CARs have not been utilized in NK-92 cells, other NK cell lines, or endogenous NK cells as other signaling domains (e.g., CD3) were deemed more efficient, especially when combined with additional signaling domains (in second and third generation CARs). Notably, the inventors have discovered that NK-92 cells expressing a first-generation CAR comprising an intracellular domain from FcεRIγ, which has only one ITAM domain, have equal or higher cytotoxic activity against cancer cells expressing the antigen recognized by the CAR than NK-92 cells expressing CARs with a CD3ζ signaling domain, which has three ITAM domains, even where these ITAM domains were combined with other signaling domains (i.e., second or third generation CARs). The inventors also made the unexpected finding that a CAR comprising an intracellular domain from FcεRIγ was expressed at higher levels on the surface of NK-92 cells than other CARs, especially those comprising the CD3ζ signaling domain. Cytotoxic effects can be even further enhanced by expression and secretion of IL-12, which reduces immune suppression in the tumor microenvironment (e.g., via exocrine stimulation of NK cells, modulation of MDSC, etc.), and/or by expression and secretion or presentation of a TGF-beta trap. Alternatively, or additionally, cytotoxic effects can also be enhanced by expression of one or more homing receptors to attract and/or retain NK cells in the tumor microenvironment.

Therefore, in some aspects of the inventive subject matter, a genetically modified NK-92 cell or NK cell line is engineered to express a chimeric antigen receptor (CAR) on a cell surface, and particularly a CAR that specifically binds to BCMA. Most typically, the CAR comprises an intracellular domain from the Fc epsilon receptor gamma (FcεRIγ) similar to the first generation construct shown in FIG. 1. However, in further contemplated embodiments the CAR may also comprise a T cell receptor (TCR) CD3 zeta (CD3ζ) intracellular domain, alone or in combination with additional components as are known from 2^{nd} and 3^{rd} generation CAR constructs (e.g., CD28, CD134, CD137, and/or ICOS). As will be readily appreciated, the CAR may be transiently or stably expressed by the NK-92 cell from a recombinant DNA or RNA molecule. Exemplary CAR constructs suitable for use herein are shown in FIG. 1.

Consequently, in one aspect of the inventive subject matter, an NK cell, an NK-92 cell or NK/NK-92 cell line expresses a chimeric antigen receptor (CAR) on the surface of the NK-92 cell that comprises a cytoplasmic domain of FcεRIγ (e.g., having amino acid sequence of SEQ ID NO:1). Alternatively, or additionally, the CAR may also comprise a cytoplasmic domain of CD3 zeta (e.g., having amino acid sequence of SEQ ID NO:7, which may be encoded by a nucleic acid of SEQ ID NO:8 (codon optimized)). In another aspect, an NK or NK-92 cell line is contemplated that is transformed with a nucleic acid encoding a chimeric antigen receptor (CAR). For example, preferred nucleic acids encode a cytoplasmic domain of FcεRIγ (e.g., comprising or consisting of SEQ ID NO:2). As discussed in more detail below, the CAR may target BCMA.

In further contemplated embodiments, the NK, NK-92, or other NK cell is modified to express an autocrine growth stimulating cytokine or variant thereof. For example, suitable cytokines may be transiently or stably expressed by the recombinant cell, and the cytokine may include an endoplasmic retention signal. Most typically (but not necessarily) a retention signal will reduce the amount of secreted cytokine and as such may act as an endocrine growth stimulus without producing systemic effects otherwise encountered by the cytokine expression. Beneficially, the nucleic acid sequence encoding the autocrine growth stimulating cytokine or variant thereof is located on the same recombinant nucleic acid, typically as part of a tri- or quadracistronic configuration. Consequently, recombinant cells transfected with the tri- or quadracistronic nucleic acid can be readily selected and propagated by virtue of their independence from otherwise needed exogenous IL-2.

Additionally, it is generally preferred that the genetically modified NK cells will also express a recombinant CD16 or high-affinity variant thereof (e.g., CD16^{158V}) to impart to the cells target specific ADCC. Advantageously, such co-expression with the CAR is thought to further increase cytotoxicity against a tumor cell. In this context, it should be appreciated that unmodified NK cells typically do not express CD16 and exhibit only cytotoxicity as a part of the innate immune system.

More recently it has been discovered that efficacy of immune therapy may be reduced or even entirely abolished by various factors present in the tumor microenvironment. For example, immune suppressive factors include among other players certain cytokines (e.g., TGF-beta) and various suppressive cells (e.g., MDSC). Consequently, genetically modified NK cells may further express one or more recombinant proteins to counteract the immune suppressive factors. For example, and as is described in more detail below, the genetically modified NK cells may express a TGF-beta trap to reduce TGF-beta mediated effects in the tumor microenvironment and/or may express IL-12 to suppress MDSCs. Additionally, or alternatively, the genetically modified NK cells may also express one or more homing receptors to the tumor microenvironment (or other desired tissue) to so increase the number of therapeutic cells in the tumor microenvironment and thus enhance the therapeutic effect.

In another aspect of the inventive subject matter, the inventors also contemplate a method of treating cancer in a patient in need thereof that includes a step of administering to the patient a therapeutically effective amount of modified NK/NK-92 cells or an NK/NK-92 cell line engineered to express a chimeric antigen receptor (CAR) as described herein. Viewed form a different perspective, the inventors also contemplate a modified NK/NK-92 cell or a NK/NK-92 cell line that expresses a chimeric antigen receptor (CAR), preferably comprising a cytoplasmic domain of FcεRIγ, for use in treating a tumor in a subject. In some embodiments, the use comprises administering to the subject effective amounts of modified cells or the cell line described herein to treat the tumor. In yet other embodiments, an in vitro method for killing tumor cells is contemplated and may include a step of contacting a tumor cell with a modified NK-92 cell or NK-92 cell line described herein. In some embodiments, the modified NK-92 cell or NK-92 cell line expresses a CAR that binds to an antigen on the tumor cell. In some embodiments, the CAR preferably comprises an intracellular domain from the Fc epsilon receptor gamma (FcεRIγ). Alternatively, or additionally, the CAR comprises a T cell receptor (TCR) CD3 zeta (CD3) intracellular domain.

With respect to suitable NK cells, it should be noted that all NK cells are deemed suitable for use herein and therefore include primary NK cells (preserved, expanded, and/or fresh cells), secondary NK cells that have been immortalized, autologous or heterologous NK cells (banked, preserved, fresh, etc.), and modified NK cells as described in more detail below. In some embodiments, it is preferred that the NK cells are NK-92 cells. The NK-92 cell line is a unique cell line that was discovered to proliferate in the presence of interleukin 2 (IL-2) (see e.g., Gong et al., Leukemia 8:652-658 (1994)). NK-92 cells are cancerous NK cells with broad anti-tumor cytotoxicity and predictable yield after expansion in suitable culture media. Advantageously, NK-92 cells have high cytolytic activity against a variety of cancers.

The original NK-92 cell line expressed the CD56^{bright}, CD2, CD7, CD11 a, CD45, and CD54 surface markers and did not display the CD1, CD3, CD4, CD5, CD8, CD10, CD14, CD16, CD19, CD20, CD23, and CD34 markers. Growth of such NK-92 cells in culture is dependent upon the presence of interleukin 2 (e.g., rIL-2), with a dose as low as 1 IU/mL being sufficient to maintain proliferation. IL-7 and IL-12 do not support long-term growth, nor have various other cytokines tested, including IL-1α, IL-6, tumor necrosis factor α, interferon a, and interferon y. Compared to primary NK cells, NK-92 typically have a high cytotoxicity even at relatively low effector:target (E:T) ratios, e.g. 1:1. Representative NK-92 cells are deposited with the American Type Culture Collection (ATCC), designation CRL-2407.

Therefore, suitable NK cells may have one or more modified KIR that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated KIR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art. Alternatively, such cells may also be commercially obtained from NantKwest (see URL www.nantkwest.com) as aNK cells (activated natural killer cells). Such cells may then be additionally genetically modified to a CAR as further described in more detail below.

In another aspect of the inventive subject matter, the genetically engineered NK cell may also be an NK-92 derivative that is modified to express the high-affinity Fcγ receptor (CD16). Sequences for high-affinity variants of the Fcγ receptor are well known in the art (see e.g., Blood 2009 113:3716-3725; SEQ ID NO:11 and SEQ ID NO:12), and all manners of generating and expression are deemed suitable for use herein. Expression of such receptor is believed to allow specific targeting of tumor cells using antibodies that are specific to a patient's tumor cells (e.g., neoepitopes), a particular tumor type (e.g., HER2/neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells. Such cells may then be additionally genetically modified to a CAR as further described in more detail below.

Therefore, NK cells suitable for use herein include NK-92 cells (which may be transfected with a tricistronic or quadracistronic construct encoding a CAR, a CD16 or variant thereof, and a cytokine or variant thereof, and optionally one of IL-12, a TGF-beta trap, and a homing receptor), a genetically modified NK cell or NK-92 cell that expresses a CD16 or variant thereof or a cytokine or variant thereof (which may be transfected with a nucleic acid encoding a CAR and a CD16 or variant thereof or a cytokine or variant thereof), and a genetically modified NK cell or NK-92 cell that expresses a CD16 or variant thereof and a cytokine or variant thereof (which may be transfected with a nucleic acid encoding a CAR). As noted before, any of the NK cells contemplated herein may express one of IL-12, a TGF-beta trap, and a homing receptor form the same or a different recombinant nucleic acid.

Genetic modification of the NK cells contemplated herein can be performed in numerous manners, and all known manners are deemed suitable for use hereon. Moreover, it should be recognized that NK cells can be transfected with DNA or RNA, and the particular choice of transfection will at least in part depend on the type of desired recombinant cell and transfection efficiency. For example, where it is desired that NK cells are stably transfected, linearized DNA may be introduced into the cells for integration into the genome. On the other hand, where transient transfection is desired, circular DNA or linear RNA (e.g., mRNA with polyA⁺ tail) may be used.

Similarly, it should be appreciated that the manner of transfection will at least in part depend on the type of nucleic acid employed. Therefore, viral transfection, chemical transfection, mechanical transfection methods are all deemed suitable for use herein. For example, in one embodiment, the vectors described herein are transient expression vectors. Exogenous transgenes introduced using such vectors are not integrated in the nuclear genome of the cell; therefore, in the absence of vector replication, the foreign transgenes will be degraded or diluted over time.

In another embodiment, vectors will preferably allow for stable transfection of cells. In one embodiment, the vector allows incorporation of the transgene(s) into the genome of the cell. Preferably, such vectors have a positive selection marker and suitable positive selection markers include any genes that allow the cell to grow under conditions that would kill a cell not expressing the gene. Non-limiting examples include antibiotic resistance, e.g. geneticin (Neo gene from Tn5). Alternatively, or additionally, the vector is a plasmid vector. In one embodiment, the vector is a viral vector. As would be understood by one of skill in the art, any suitable vector can be used, and suitable vectors are well-known in the art.

In still other embodiments, the cells are transfected with mRNA encoding the protein of interest (e.g., the CAR). Transfection of mRNA results in transient expression of the protein or proteins. In one embodiment, transfection of mRNA into NK-92 cells is performed immediately prior to administration of the cells. In one embodiment, “immediately prior” to administration of the cells refers to between about 15 minutes and about 48 hours prior to administration. Preferably, mRNA transfection is performed about 5 hours to about 24 hours prior to administration. In at least some embodiments as described in more detail below, NK cell transfection with mRNA resulted in unexpectedly consistent and strong expression of the CAR at a high faction of transfected cells. Moreover, such transfected cells also exhibited a high specific cytotoxicity at comparably low effector to target cell ratios.

With respect to contemplated CARs it is noted that the NK/NK-92 cells will be genetically modified to express the CAR as a membrane bound protein exposing a portion of the CAR on the cell surface while maintaining the signaling domain in the intracellular space. Most typically, the CAR will include at least the following elements (in order): an extracellular binding domain, a hinge domain, a transmembrane domain, and a signaling domain (preferably, but not necessarily a FcεRIγ domain).

In preferred embodiments, the cytoplasmic domain of the CAR comprises or consists of a signaling domain of FcεRIγ. For example, the FcεRIγ signaling domain comprises or consists of or consists essentially of the amino acid sequence of SEQ ID NO:1. In some embodiments, the FcεRIγ cytoplasmic domain is the sole signaling domain. However, it should be appreciated that additional elements may also be included, such as other signaling domains (e.g., CD28 signaling domain, CD3ζ signaling domain, 4-1BB signaling domain, etc.). These additional signaling domains may be positioned downstream of the FcεRIγ cytoplasmic domain and/or upstream of the FcεRIγ cytoplasmic domain.

In some embodiments, the FcεRIγ signaling domain comprises or consists of or consists essentially of an amino acid sequence having at least about 85%, 90%, 95%, 96%, 97%, 98%, or 99% sequence homology to the amino acid sequence of SEQ ID NO:1.

As noted above, in some embodiments, the cytoplasmic domain of the CAR comprises a signaling domain of CD3 zeta (CD3ζ). In one embodiment, the cytoplasmic domain of the CAR consists of a signaling domain of CD3 zeta. In one embodiment, the CD3 zeta signaling domain comprises or consists of or consists essentially of the amino acid sequence of SEQ ID NO:7. In some embodiments, the CD3 zeta signaling domain comprises or consists of or consists essentially of an amino acid sequence having at least about 85%, 90%, 95%, 96%, 97%, 98%, or 99% sequence homology to the amino acid sequence of SEQ ID NO:7.

The CAR may comprise any suitable transmembrane domain. In one aspect, the CAR comprises a transmembrane domain of CD28. In one embodiment, the CD28 transmembrane domain comprises or consists of or consists essentially of the amino acid sequence of SEQ ID NO:4. In one embodiment, the CD28 transmembrane domain comprises or consists of or consists essentially of an amino acid sequence having at least about 85%, 90%, 95%, 96%, 97%, 98%, or 99% sequence homology to the amino acid sequence of SEQ ID NO:4. In one embodiment, the transmembrane domain is selected from a CD28 transmembrane domain, 4-1BB transmembrane domain, or FcεRIγ transmembrane domain.

The CAR may comprise any suitable hinge region. In one aspect, the CAR comprises a hinge region of CD8. In one embodiment, the CD8 hinge region comprises or consists of or consists essentially of the amino acid sequence of SEQ ID NO:3. In one embodiment, the CD8 hinge region comprises or consists of or consists essentially of an amino acid sequence having at least about 85%, 90%, 95%, 96%, 97%, 98%, or 99% sequence homology to the amino acid sequence of SEQ ID NO:3.

Most typically, but not necessarily, the extracellular binding domain of the CAR will be a scFv or other natural or synthetic binding portion that specifically binds a B-cell maturation antigen. Especially suitable binding portions include small antibody fragments with single, dual, or multiple target specificities, beta barrel domain binders, page display fusion proteins, etc. However, in other embodiments, suitable extracellular binding domains will specifically bind to a tumor-specific antigen, a tumor associated antigen, or a patient- and tumor-specific antigen. In one particularly preferred embodiment, the contemplated antigen comprise BCMA. Other contemplated antigens may include CD19, CD20, GD2, CD30, FAP, CD33, CD123, PD-L1, IGF1R, CSPG4, or B7-H4. Further tumor-specific antigens are described, by way of non-limiting example, in US2013/0189268; WO 1999024566 A1; US 7098008; and WO 2000020460, each of which is incorporated herein by reference in its entirety.

Therefore, contemplated CARs will generally have a structure of an extracellular binding domain that is (directly) coupled to a hinge domain, which is (directly) coupled to a transmembrane domain, which is (directly) coupled to a (e.g., FcεRIγ) signaling domain. In still further contemplated aspects, contemplated CARs may also include one or more signaling domains in addition to or replacing the FcεRIγ signaling domain, and especially contemplated signaling domains include CD3ζ signaling domains, 4-1BB signaling domains, and CD28 signaling domains. In one example, contemplated CARs may comprise a BCMA CAR having a nucleic acid sequence of SEQ ID NO:38, or a sequence having identity to SEQ ID NO:38 of at least 90%, or at least 95%, or at least 96%, or at least 97%, or at least 98%, or at least 99%. In another example, contemplated CARs may comprise a BCMA CAR having a BCMA VL-VH scFv, and a nucleic acid sequence of SEQ ID NO:39, or a polypeptide sequence of SEQ ID NO:41, or a sequence having identity to SEQ ID NO:39 or SEQ ID NO:41 of at least 90%, or at least 95%, or at least 96%, or at least 97%, or at least 98%, or at least 99%. In another example, contemplated CARs may comprise a BCMA CAR having a BCMA VH-VL scFv, and a nucleic acid sequence of SEQ ID NO:40, or a polypeptide sequence of SEQ ID NO:42, or a sequence having identity to SEQ ID NO:40 or SEQ ID NO:42 of at least 90%, or at least 95%, or at least 96%, or at least 97%, or at least 98%, or at least 99%.

In another example, contemplated CARs may therefore include any one of the binding domains having SEQ ID NOs:39-42 or a sequence having identity to SEQ ID NO:39 or SEQ ID NO:40 or SEQ ID NO:41 or SEQ ID NO:42 of at least 90%, or at least 95%, or at least 96%, or at least 97%, or at least 98%, or at least 99%. that is coupled to a hinge domain (e.g., CD8 hinge as in SEQ ID NO:3), which is in turn coupled to a transmembrane domain (e.g., CD28 TM as in SEQ ID NO:4), which is coupled to a signaling domain (e.g., FcεRIγ signaling domain as in SEQ ID NO:1, CD28 signaling domain as in SEQ ID NO:5, 4-1BB signaling domain as in SEQ ID NO:6, and/or CD3ζ signaling domain as in SEQ ID NO:7)

With respect to the construction of contemplated CARs it should be recognized that CARs can be engineered in numerous manners as described, for example, in WO 2014/039523; US 2014/0242701; US 2014/0274909; US 2013/0280285 and WO 2014/099671, each of which is incorporated herein by reference in its entirety.

Viewed from a different perspective, contemplated CARs target an antigen associated with a specific cancer type, and more particularly cancers that overexpress BCMA, such as multiple myeloma, or Hodgkin's lymphoma.

In still further contemplated aspects, NK cells may be further genetically modified to express one or more cytokines, and especially an autocrine growth stimulating cytokine to so provide a selection marker where the cytokine and the CAR are encoded on the same recombinant nucleic acid and/or to render the recombinant cells independent of exogenous IL-2. Therefore, in some embodiments, NK-92 cells are modified to express at least one cytokine. In particular, the at least one cytokine is IL-2, IL-12, IL-15, IL-18, IL-21, or a variant thereof. In preferred embodiments, the cytokine is IL-2 or a variant thereof and especially preferred variants include endoplasmic retention signals (e.g., human IL-2 as in SEQ ID NO:9, optionally with ER retention signal as in SEQ ID NO:10). For example, the IL-2 gene is cloned and expressed with a signal sequence that directs the IL-2 to the endoplasmic reticulum. This permits expression of IL-2 at levels sufficient for autocrine activation, but without releasing IL-2 extracellularly (e.g., Exp Hematol. 2005 Feb;33(2):159-64.) Alternatively, expression of a cytokine (and especially IL-15) may also be such that the cytokine will be expressed in sufficient quantities to provide an autocrine growth signal to the recombinant cells, but also to allow at least some of the expressed IL-15 to be released from the cell, which will so provide an immune stimulatory signal. For example, such expression may be achieved using a human IL-15 sequence that includes both the signal peptide and an endoplasmic retention sequence. An exemplary DNA and protein sequence for an endoplasmic retained IL-15 is shown in SEQ ID NO:36 and SEQ ID NO:37, respectively.

Where desired, contemplated cells may also express a suicide gene. The term “suicide gene” refers to a transgene that allows for the negative selection of cells expressing the suicide gene. A suicide gene is used as a safety system, allowing cells expressing the gene to be killed by introduction of a selective agent. This is desirable in case the recombinant gene causes a mutation leading to uncontrolled cell growth, or the cells themselves are capable of such growth. A number of suicide gene systems have been identified, including the herpes simplex virus thymidine kinase (TK) gene, the cytosine deaminase gene, the varicella-zoster virus thymidine kinase gene, the nitroreductase gene, the Escherichia colt gpt gene, and the E. coli Deo gene. Typically, the suicide gene encodes for a protein that has no ill effect on the cell but, in the presence of a specific compound, will kill the cell. Thus, the suicide gene is typically part of a system.

In one embodiment, the suicide gene is active in NK-92 cells. In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir. In another embodiment, the suicide gene is cytosine deaminase, which is toxic to cells in the presence of 5-fluorocytosine. Garcia-Sanchez et al. “Cytosine deaminase adenoviral vector and 5-fluorocytosine selectively reduce breast cancer cells 1 million-fold when they contaminate hematopoietic cells: a potential purging method for autologous transplantation.” Blood. 1998 Jul. 15; 92(2):672-82. In a further embodiment, the suicide gene is cytochrome P450, which is toxic in the presence of ifosfamide or cyclophosphamide. See, e.g. Touati et al. “A suicide gene therapy combining the improvement of cyclophosphamide tumor cytotoxicity and the development of an anti-tumor immune response.” Curr Gene Ther. 2014;14(3):236-46. In yet another embodiment, the suicide gene is iCasp9. Di Stasi, (2011) “Inducible apoptosis as a safety switch for adoptive cell therapy.” N Engl J Med 365: 1673-1683. See also Morgan, “Live and Let Die: A New Suicide Gene Therapy Moves to the Clinic” Molecular Therapy (2012); 20: 11-13. iCasp9 induces apoptosis in the presence of a small molecule, AP1903. AP1903 is biologically inert small molecule, that has been shown in clinical studies to be well tolerated, and has been used in the context of adoptive cell therapy.

Where the modified NK cells are further engineered to express IL-12, it is generally preferred that the IL-12 is expressed as a single chain heterodimer in which the p35 and p40 components are linked together by a flexible linker (in either orientation, p35-linker-p40 or p40-linker-p35). Moreover, it is generally preferred that the heterodimer will be secreted and as such may include a signal peptide for protein export. Therefore, suitable IL-12 sequences as contemplated herein may comprise a nucleic acid sequence with at least 80%, 85%, 90%, 91%, 92%, 93%, 94%, 95%, 96%, 97%, 98% or 99% identity to SEQ ID NO:26 (p35 n.t. sequence), or SEQ ID NO:28 (p40 n.t. sequence). The IL-12 contemplated herein may also comprise an amino acid sequence with at least 80%, 85%, 90%, 91%, 92%, 93%, 94%, 95%, 96%, 97%, 98% or 99% identity to SEQ ID NO:27 (p35 a.a. sequence, isoform 1 precursor), or SEQ ID NO:29 (p40 a.a. sequence, precursor). Thus, the IL-12 single chain p40 p35 sequence may comprise a polypeptide sequence with at least 80%, 85%, 90%, 91%, 92%, 93%, 94%, 95%, 96%, 97%, 98% or 99% identity to SEQ ID NO:30, or may comprise an polynucleotide sequence with at least 80%, 85%, 90%, 91%, 92%, 93%, 94%, 95%, 96%, 97%, 98% or 99% identity to SEQ ID NO:31. Most preferably, but not necessarily, the nucleic acid encoding the IL-12 single chain heterodimer will be part of a polycistronic nucleic acid sequence (e.g., present as a quadracistronic sequence with CAR, CD16, and erIL-2).

Where the modified NK cells are further engineered to express a TGF-beta trap, it is generally preferred that the TGF-beta trap is a single chain dimer of the extracellular domain of a TGFβRII molecule, and most preferably comprises a single chain dimer of the TGF-beta Receptor II ectodomain. Suitable TGF-beta traps are therefore encoded by a polynucleotide sequence with at least 80%, 85%, 90%, 91%, 92%, 93%, 94%, 95%, 96%, 97%, 98% or 99% identity to SEQ ID NO:32 (TGFBRII extracellular domain), or SEQ ID NO:34 (TGF beta trap sequence). The TGF-beta trap contemplated herein may also comprise an amino acid sequence with at least 80%, 85%, 90%, 91%, 92%, 93%, 94%, 95%, 96%, 97%, 98% or 99% identity to SEQ ID NO:33 (TGFBRII extracellular domain), or SEQ ID NO:35(TGF-beta trap sequence). Other suitable TGF-beta traps include those described in Mol. Canc. Ther. 2012, Vol 11(7), 1477-1487. Most preferably, but not necessarily, the nucleic acid encoding the TGF-beta trap will be part of a polycistronic nucleic acid sequence (e.g., present as a quadracistronic sequence with CAR, CD16, and erIL-2).

Where the modified NK cells are further engineered to express a homing receptor it is noted that the term “homing receptor” refers to a receptor that activates a cellular pathway that results directly or indirectly in the cell migrating toward a target cell or tissue. For example, homing receptors expressed by leukocytes are used by leukocytes and lymphocytes to enter secondary lymphoid tissues via high endothelial venules. Homing receptors can also be used by cells to migrate toward the source of a chemical gradient, such as a chemokine gradient. Examples of homing receptors include G-protein coupled receptors such as chemokine receptors, including CCR1, CCR2, CCR3, CCR4, CCRS, CCR6, CCR7, CCR8, CCR9, CCR10, CXCR1, CXCR2, CXCR3, CXCR4, CXCRS, CXCR6, CXCR7, CX3CR1, XCR1, CCXCKR, D6, and DARC; cytokine receptors; cell adhesion molecules such as selectins, including L-selectin (CD62L), integrins such as a4(37 integrin, LPAM-1, and LFA-1. Homing receptors generally bind to cognate ligands on the target tissues or cell. In some embodiments, homing receptors bind to addressins on the endothelium of venules, such as mucosal vascular addressing cell adhesion molecule 1 (MAdCAM-1).

In some exemplary embodiments, the chemokines and homing receptors contemplated herein may comprise a polypeptide sequence or a polynucleotide sequence having at least 80%, 85%, 90%, 91%, 92%, 93%, 94%, 95%, 96%, 97%, 98% or 99% identity to SEQ ID NO:13 (CCR7 nt. sequence), or SEQ ID NO:14 (CCL19 n.t. sequence), or SEQ ID NO:15 (CCL21 n.t. sequence), or SEQ ID NO:16 (CXCR2 n.t. sequence), or SEQ ID NO:17 (CXCR2 a.a. sequence), or SEQ ID NO:18 (CXCL4 n.t. sequence), or SEQ ID NO:19 (CXCL4 a.a. sequence), or SEQ ID NO:20 (CD62L n.t. sequence), or SEQ ID NO:21 (CD62L a.a. sequence), or SEQ ID NO:22 (IL-8 n.t. sequence), or SEQ ID NO:23 (IL-8 a.a. sequence), or SEQ ID NO:24 (CXCL1 n.t. sequence), or SEQ ID NO:25 (CXCL1 a.a. sequence). Most preferably, but not necessarily, the nucleic acid encoding the homing receptor will be part of a polycistronic nucleic acid sequence (e.g., present as a quadracistronic sequence with CAR, CD16, and erIL-2).

Of course, it should be noted that all of the recombinant proteins can be expressed from individual recombinant sequences. However, it is generally preferred that where multiple recombinant sequences are expressed (e.g., CAR, CD16, cytokine, TGF-beta trap), coding regions may be arranged in a polycistronic unit with at least two or at least three or at least four coding regions encoding the recombinant proteins. Therefore, transgenes can be engineered into an expression vector by any mechanism known to those of skill in the art. Where multiple transgenes are to be inserted into a cell, transgenes may be engineered into the same expression vector or a different expression vector. In some embodiments, the cells are transfected with mRNA encoding the transgenic protein to be expressed. In some embodiments, the cells are transfected with DNA encoding the transgenic protein to be expressed. Transgenes, mRNA and DNA can be introduced into the NK-92 cells using any transfection method known in the art, including, by way of non-limiting example, infection, viral vectors, electroporation, lipofection, nucleofection, or “gene-gun.”

In preferred embodiments, it should therefore be noted that the genetically modified NK cell (especially where the cell expresses a CAR and CD16 or variant thereof) will exhibit three distinct modes of cell killing: General cytotoxicity which is mediated by activating receptors (e.g., an NKG2D receptor), ADCC which is mediated by antibodies bound to a target cell, and CAR mediated cytotoxicity. Where desired, therapeutic effect in the tumor microenvironment may be further increased via expression and secretion of IL-12 (e.g., as a single chain heterodimer), via expression and presentation/secretion of a TGF-beta trap (e.g., as a single chain dimer of the TGF-beta Receptor II ectodomain) or via expression of a homing receptor (e.g., CCR7). As will be readily apparent, contemplated genetically modified cells can be used for treatment of various diseases, and especially of various cancers and viral infections where a diseased cell presents a disease-specific or disease-associated antigen. Consequently, the inventors contemplate methods of treating patients with modified NK or NK-92 cells as described herein. In one embodiment, the patient is suffering from cancer (e.g., a tumor) and the modified NK-92 cell or cell line expresses a CAR specific for an antigen expressed on the surface of a cell from the cancer or tumor. As noted above, in some embodiments, the cancer may be multiple myeloma or Hodgkin's lymphoma.

Contemplated modified NK or NK-92 cells can be administered to an individual by absolute numbers of cells. For example, the individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) modified NK-92 cells per injection, or any ranges between any two of the numbers, end points inclusive. In other embodiments, modified NK-92 cells can be administered to an individual by relative numbers of cells, e.g., said individual can be administered about 1000 cells to up to about 10 billion cells per kilogram of the individual, such as at about, at least about, or at most about, 1×10⁸, 1×10⁷, 5 ×10⁷, 1×10⁶, 5 ×10⁶, 1×10⁵, 5 ×10⁵, 1 ×10⁴, 5 ×10⁴, 1 ×10³, 5 ×10³ (and so forth) modified NK-92 cells per kilogram of the individual, or any ranges between any two of the numbers, end points inclusive. In other embodiments, the total dose may be expressed in m² of body surface area, including about 1×10¹¹ 1×10¹⁰, 1×10⁹, 1×10⁸, 1×10⁷, per m², or any ranges between any two of the numbers, end points inclusive. The average person is about 1.6 to about 1.8 m². In a preferred embodiment, between about 1 billion and about 3 billion NK-92 cells are administered to a patient.

Modified NK-92 cells, and optionally other anti-cancer agents can be administered once to a patient with cancer or infected with a virus or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

In one embodiment, where the modified NK cells express a suicide gene, the patient is administered an agent to trigger modified NK cell death. In one embodiment, the agent is administered at a time point after administration of the modified NK cells that is sufficient for the NK cells to kill target cells.

In one embodiment, the modified NK cells are irradiated prior to administration to the patient. Irradiation of NK cells is described, for example, in U.S. Pat. No. 8,034,332, which is incorporated herein by reference in its entirety. In one embodiment, modified NK cells that have not been engineered to express a suicide gene are irradiated.

Furthermore, it should be appreciated that contemplated treatments will also include administration of other immune therapeutic entities, and especially preferred immune therapeutic entities include a viral cancer vaccine (e.g., adenoviral vector encoding cancer specific antigens), a bacterial cancer vaccine (e.g., non-pyrogenic E. coli expressing one or more cancer specific antigens), a yeast cancer vaccine, N-803 (also known as ALT-803, ALTOR Biosciences), an antibody (e.g., binding to a tumor associated antigen or patient specific tumor neoantigen), a stem cell transplant (e.g., allogeneic or autologous), and a tumor targeted cytokine (e.g., NHS-IL12, IL-12 coupled to a tumor targeting antibody or fragment thereof).

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before aspects of the present inventive subject matter are disclosed and described in more detail, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1

### B-Cell Maturation Antigen (BCMA) CAR Construct

Unless specified otherwise in the following examples, the inventors used NK-92 cells for all transfections of the cells with recombinant nucleic acids. Furthermore, and also unless noted otherwise, all recombinant nucleic acids were linearized DNA constructs encoding a tri- or quadracistronic configuration.

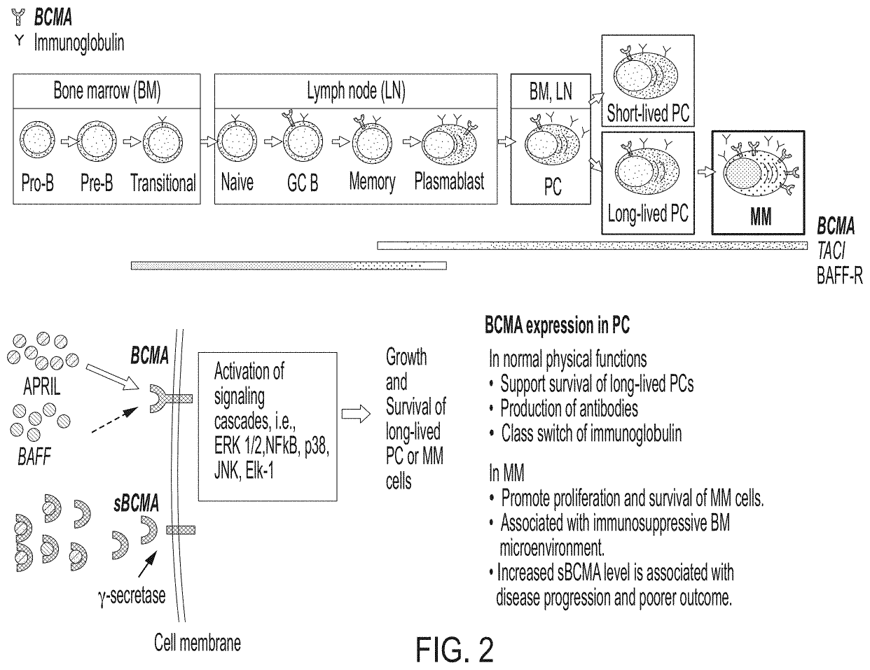

B-cell maturation antigen (BCMA or BCM), also known as tumor necrosis factor receptor superfamily member 17 (TNFRSF17), is a protein that in humans is encoded by the TNFRSF17 gene. This receptor is preferentially expressed in mature B lymphocytes, and has been shown to specifically bind to the tumor necrosis factor (ligand) superfamily, and to lead to NF-kappaB and MAPK8/JNK activation. In normal physical functions, BCMA expression in plasma cells (PC) support survival of long lived PCs, production of antibodies, and class switch of immunoglobulin. In multiple myeloma (MM), BCMA promotes proliferation and survival of MM cells, is associated with immunosuppressive BM microenvironment, and increased sBCMA level is associated with disease progression and poorer outcome. This background of BMCA is further illustrated in FIG. 2

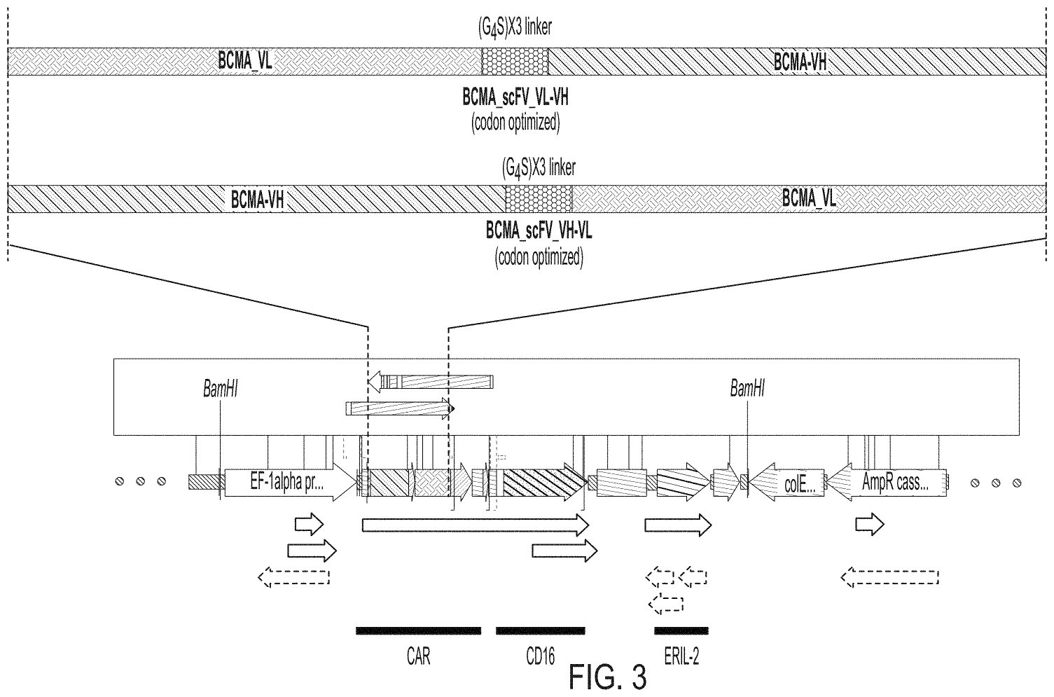

To generate BCMA.CAR t-haNK cells, a recombinant DNA molecule was assembled as is schematically depicted in FIG. 3 where the tricistronic configuration included a sequence encoding a BCMA.CAR followed by a P2A sequence, which was followed by a sequence encoding CD16 (or CD16^{158V}), and which in turn was followed by an IRES sequence element upstream of a sequence encoding erIL-2. The primary transcript of the tricistronic nucleic acid then lead to the formation of BCMA.CAR, CD16^{158V}, and erIL-2 as the recombinant polypeptides. FIG. 3 exemplarily and schematically depicts two linearized recombinant nucleic acid used for transfection of the NK-92 cells. As illustrated in FIG. 3, the BCMA.CAR construct may comprise a VH chain followed by a VL, or VL followed by VH. Typically, VH and VL are linked by a 15 amino acid linker, such as (G₄S)₃.

All transfections generally followed standard protocol: NK-92 cells were grown in X-Vivo10 medium (Lonza, Basel, Switzerland) supplemented with 5% Human AB Serum (Valley Biomedical, Winchester, Va.) and 500 IU/mL IL-2 (Prospec, Rehovot, Israel). Cells were electroporated with tricistronic or quadracistronic DNA using the Neon™ electroporation device (Life Technologies, Carlsbad, Calif.), following the manufacturer's parameters for NK-92 cells (1250 V, 10 ms, 3 pulses) and using 2μg of DNA per 10⁶ cells in a volume of 100 μl. Electroporated cells were transferred to IL-2 free medium (same as above).

Selection for recombinant cells and clones was based on continued cell culture in the absence of exogenously added IL-2 as all recombinant cells included a recombinant autocrine growth promoting cytokine (e.g., IL-2, erIL-2, IL-15, or erIL-15).

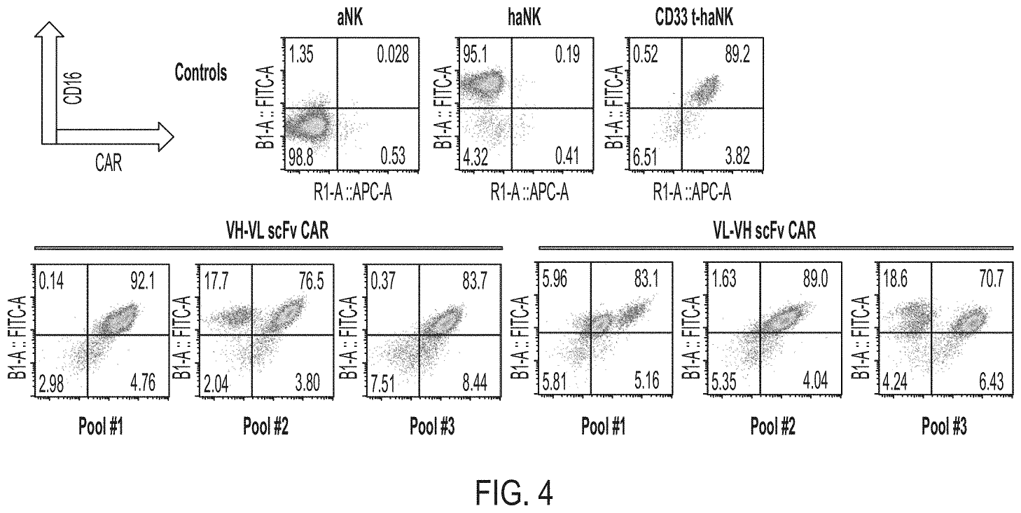

The BCMA.CAR and CD16 expression on the NK-92 cell surface was determined by flow cytometry using a biotinylated anti-scFv antibody and streptavidin labeled with allophycocyanin, and fluorescently labeled anti-CD16 antibody. Exemplary results for BCMA.CAR and CD16 expression (polyclonal) are shown in FIG. 4 along with aNK (not expressing CD16, not expressing BCMA.CAR) and haNK (expressing CD16, but not expressing BCMA.CAR) controls. As can be readily seen from FIG. 4, the polyclonal cell cultures had a significant and strong expression for both, BCMA.CAR and CD16^{158V}. Moreover, it should be noted that the BCMA.CAR expressed at relatively the same level irrespective of whether the BCMA construct used as VL-VH or VH-VL orientation.

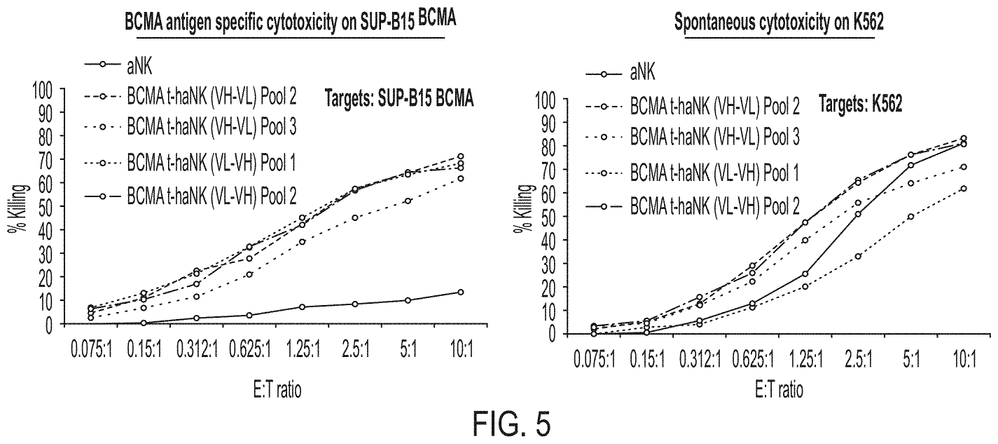

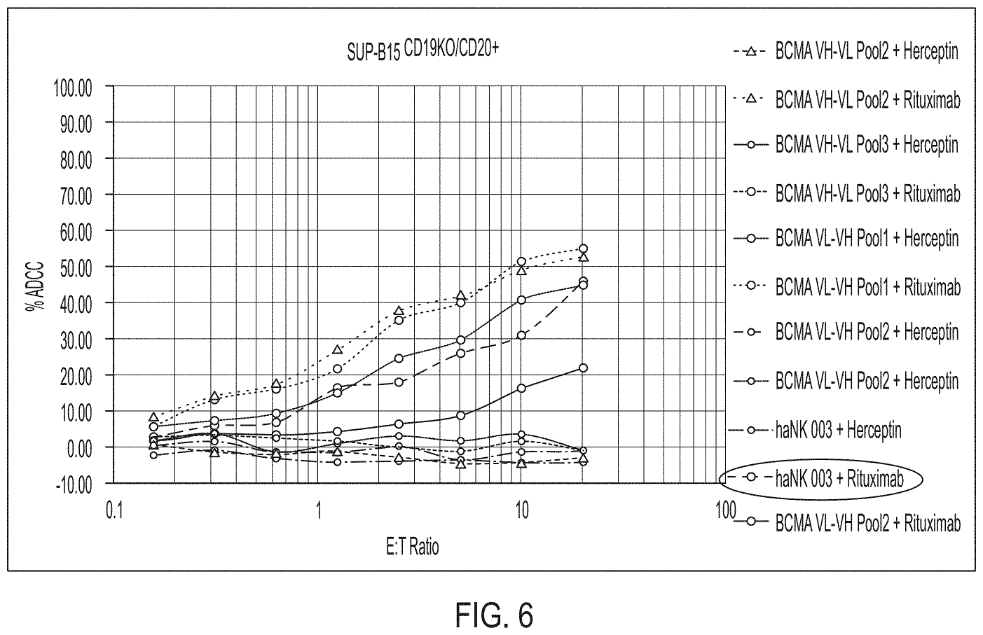

BCMA.CAR t-haNK cells had strong and target specific CAR-mediated cytotoxicity as can be seen from the results depicted in FIG. 5. The left panel of FIG. 5 is CAR-mediated toxicity against SUP-B15 BCMA+target cells, while right panel shows spontaneous cytotoxicity against K562 target cells. ADCC was tested using SUP-B15^{HER-2-/CD20+} cells and rituximab as target specific antibody and Herceptin as control antibody as shown in FIG. 6. Once again, the polyclonal BCMA.CAR t-haNK cells had strong and target specific ADCC approaching about 55% as can be seen from the exemplary results depicted in FIG. 6, with no substantial off-target toxicity. The results shown in FIGS. 4-6 are with respect to transfection pools (pools VH-VL # 1 to 3 and VL-VH # 1 to 3) of the BCMA.CAR t-haNK cells. Upon getting successful results from these pools, the inventors made clones from the BCMA.CAR t-haNK cells in these pools. The results from these clones are described below.

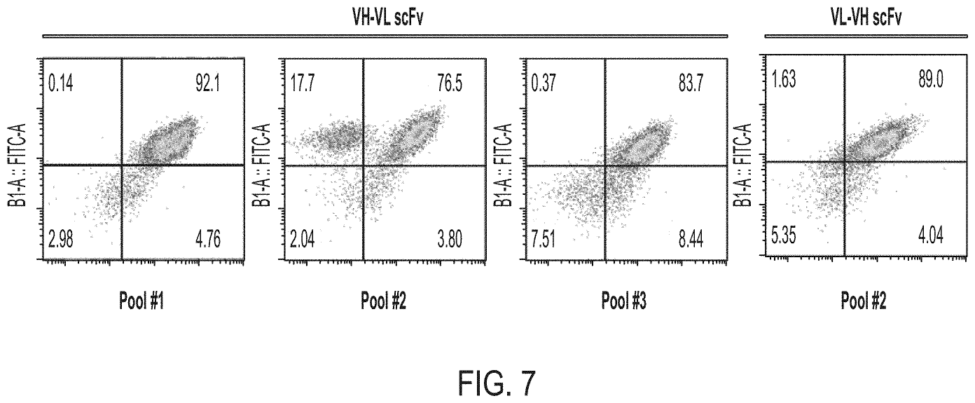

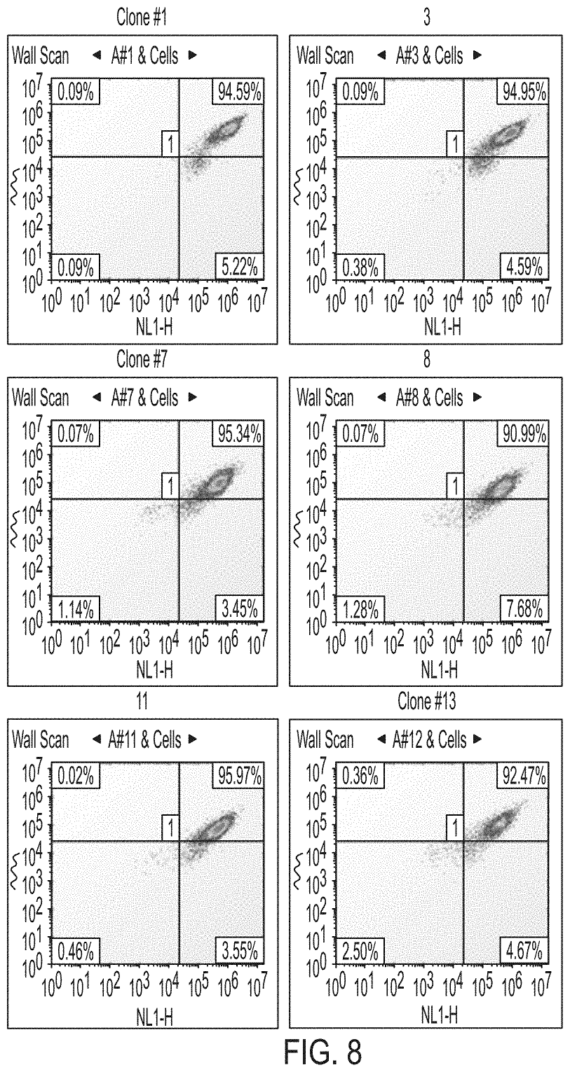

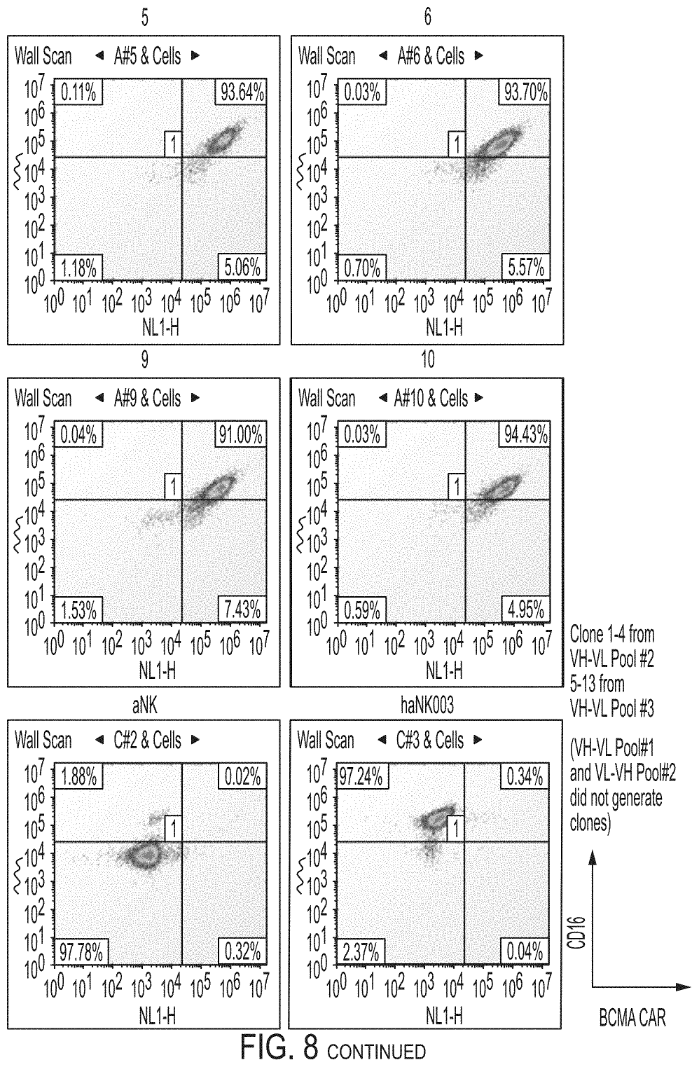

A number of individual clones from the BCMA.CAR t-haNK cell population were prepared following dilution propagation as illustrated in FIG. 7 and FIG. 8 respectively. FIG. 7 depicts CAR expression on transfection pools VH-VL # 1 to 3 and VL-VH # 2. Clones were prepared from these transfection pools by limiting dilution, and CAR expression analysis of the clones were done, as illustrated in FIG. 8. Expression analysis was performed via FACS using the same procedure as described above. aNK and haNK cells were used as controls. It was observed that most individual clones had a significant and strong expression of both BCMA.CAR and CD16^{158V} as can be seen from the exemplary results of FIG. 8. However, a few of the clones, # 2, # 4, and # 14 did not have significant and strong expression of either BCMA.CAR or CD16^{158V}, or both.

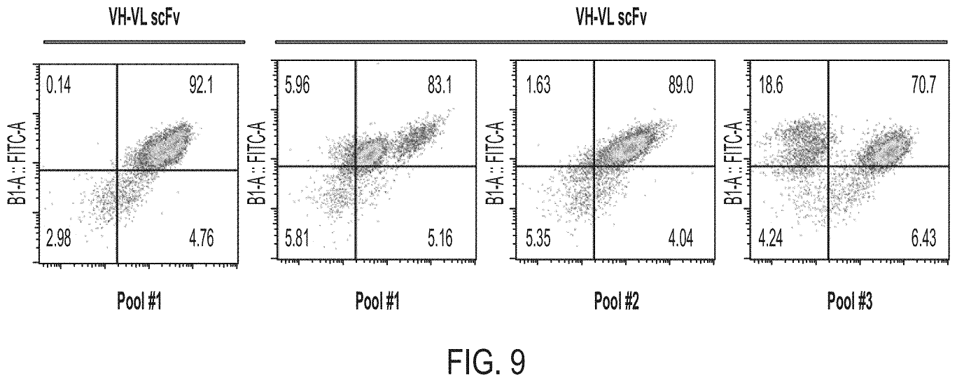

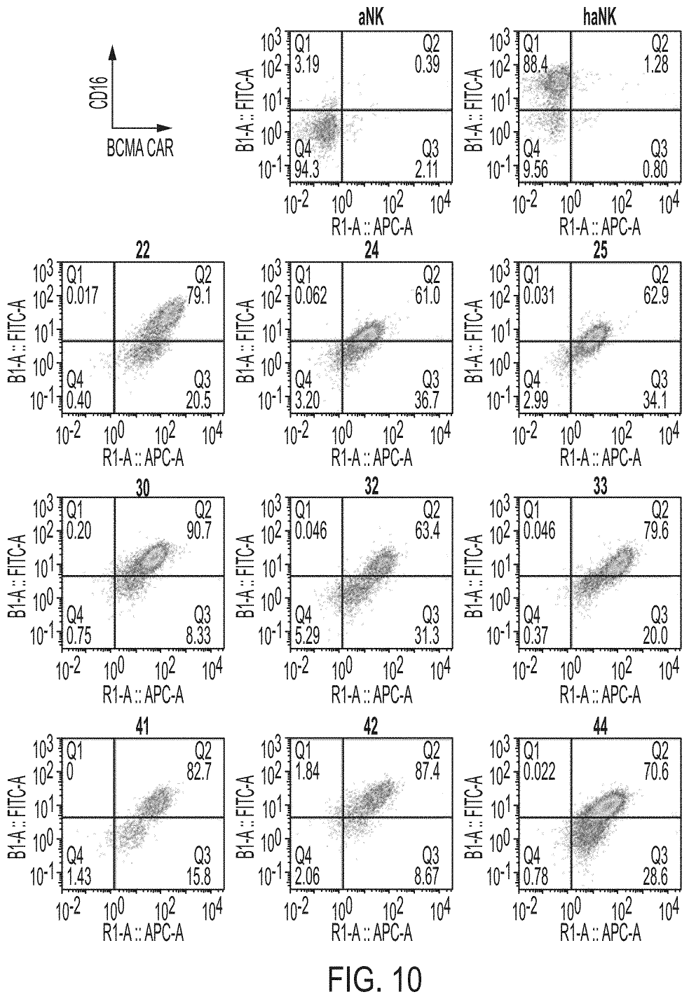

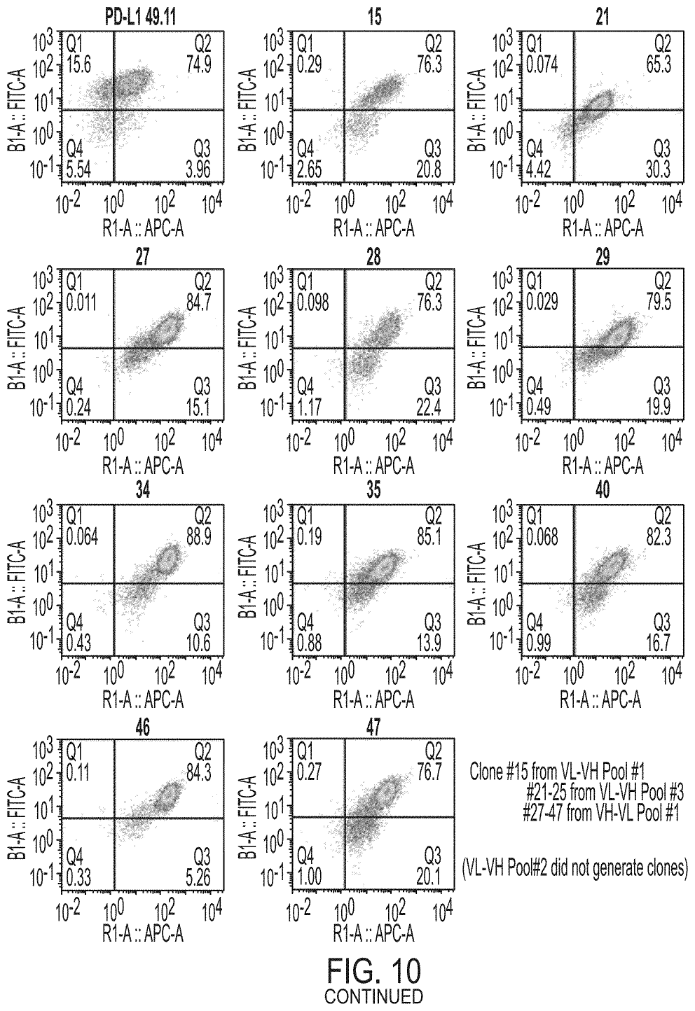

In another embodiment, individual clones from the BCMA.CAR t-haNK cell populations were also prepared following dilution propagation as illustrated in FIG. 9 and FIG. 10 respectively. FIG. 9 depicts CAR expression on transfection pools VL-VH # 1 to 3 and VH-VL # 2 respectively. Expression analysis was performed via FACS using the same procedure as described above. aNK and haNK cells were used as controls. Again, all clones obtained by limiting dilution were observed to have a significant and strong expression of both BCMA.CAR and CD16^{158V} as can be seen from the exemplary results of FIG. 10. It should be noted that clone # 15 is from VL-VH pool # 1; clones 21-25 are from VL-VH pool # 3; and clones 27-47 are from VH-VL pool # 1.

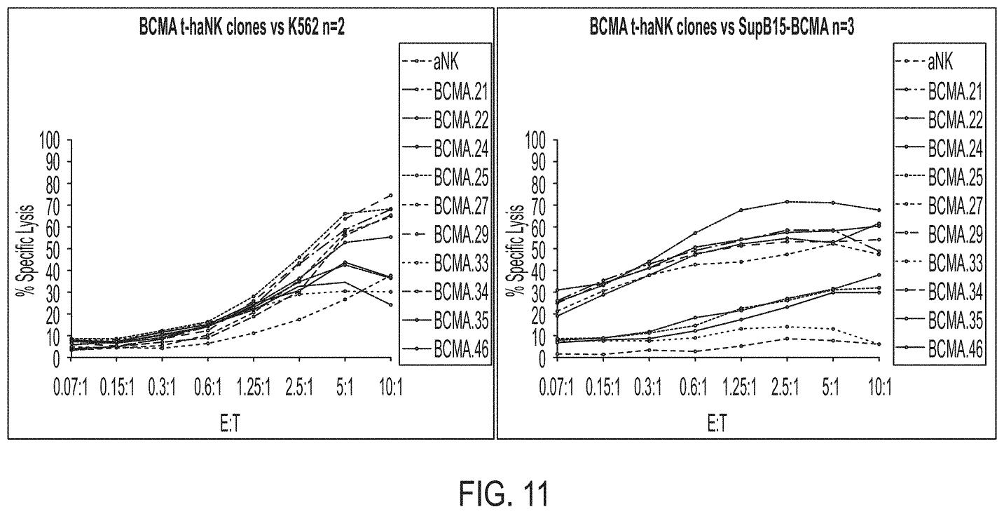

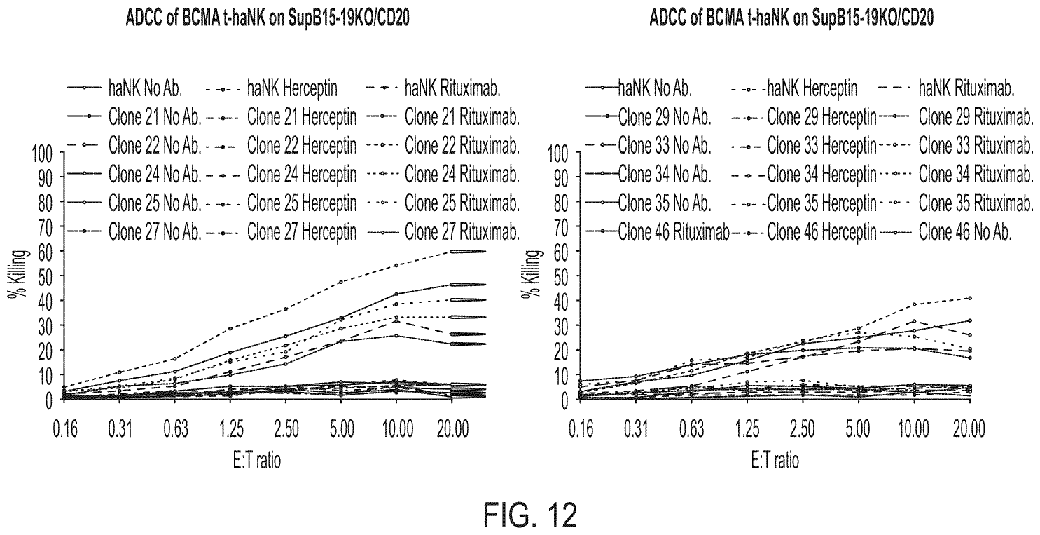

Individual BCMA.CAR t-haNK tricistronic cell clones were tested for CAR and natural cytotoxicity as illustrated in FIG. 11. As can be seen from FIG. 11, most of the of BCMA tricistronic clones maintain the CAR and natural cytotoxicity that was seen in the transfection pools. ADCC of BCMA.CAR t-haNK tricistronic clones were done on SUP-B15-19KO/CD20 cells, as illustrated in FIG. 12. FIG. 12 illustrates that most of the of BCMA tricistronic clones maintain the ADCC that was seen in the transfection pools. Furthermore, growth rates of the clones are shown below in Table 1. As can be seen from Table 1, the growth rate of most of the BCMA tricistronic clones are comparable to the growth rate of the aNK cells.

TABLE 1

| | Average growth hr(−1) |
|---|---|
| aNK | 38.5 |
| BCMA.21 | 40.7 |
| BCMA.22 | 41.1 |
| BCMA.24 | 56.4 |
| BCMA.25 | 51.2 |
| BCMA.27 | 41.3 |
| BCMA.29 | 44.9 |
| BCMA.33 | 43.3 |
| BCMA.34 | 58.1 |
| BCMA.35 | 34.9 |
| BCMA.46 | 42.5 |

### Example 2

### Quadracistronic Constructs

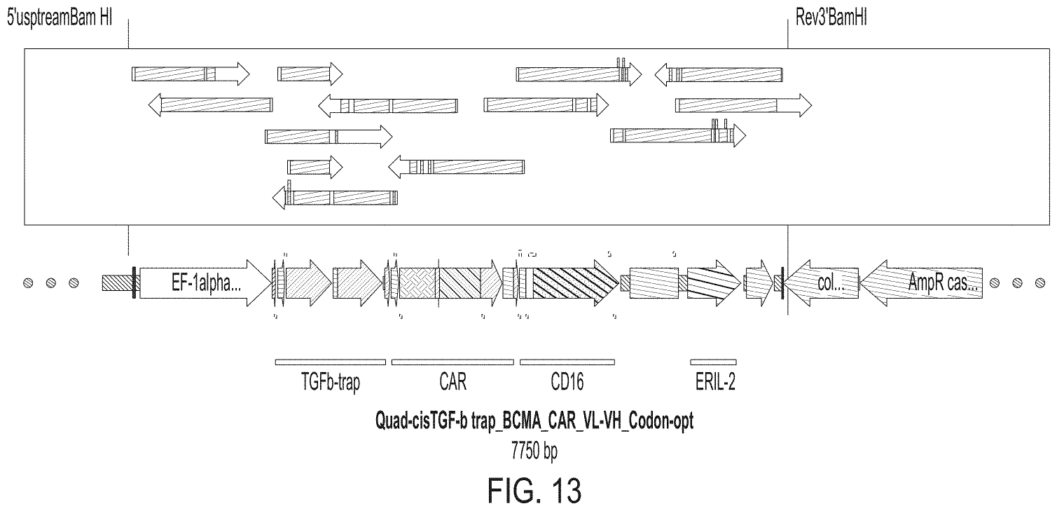

While the above examples were performed with tricistronic constructs it should be appreciated that the same constructs can also be implemented in a quadracistronic construct to express IL-12, a TGF-beta trap, or a homing receptor to so reduce immune suppression in the tumor microenvironment and enrich the tumor microenvironment with thusly modified NK cells. One exemplary quadracistronic construct is schematically illustrated in FIG. 13 where a nucleic acid sequence encoding a TGF-beta trap is upstream of the tricistronic construct as discussed above. A P2A sequence may be located between the TGF-beta trap and the CAR to ensure coordinated expression while producing distinct proteins. Expression of the TGF-beta trap was then tested in an ELISA to ascertain that the quadracistronic construct indeed produced a functional TGF-beta trap. Notably, expression levels of the TGF-beta trap were significant throughout all recombinant clones with quadracistronic construct.

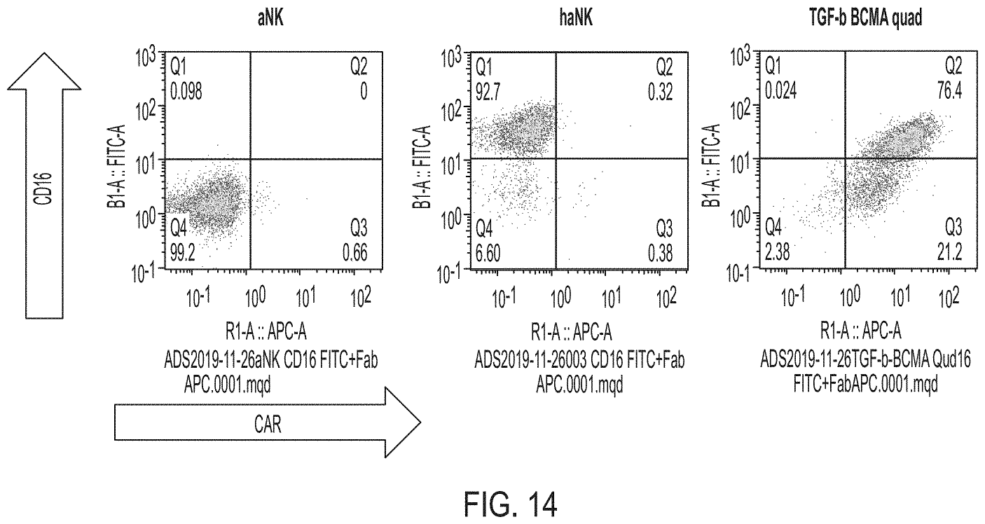

The BCMA.CAR and CD16 expression on the cell surface of NK-92 cells having the quadracistronic vector was determined by flow cytometry using biotinylated anti-scFv antibody and streptavidin labeled with allophycocyanin, and fluorescently labeled anti-CD16 antibody. Exemplary results for BCMA.CAR and CD16 expression (polyclonal) are shown in FIG. 14 along with aNK (not expressing CD16, not expressing BCMA.CAR) and haNK (expressing CD16, but not expressing BCMA.CAR) controls.

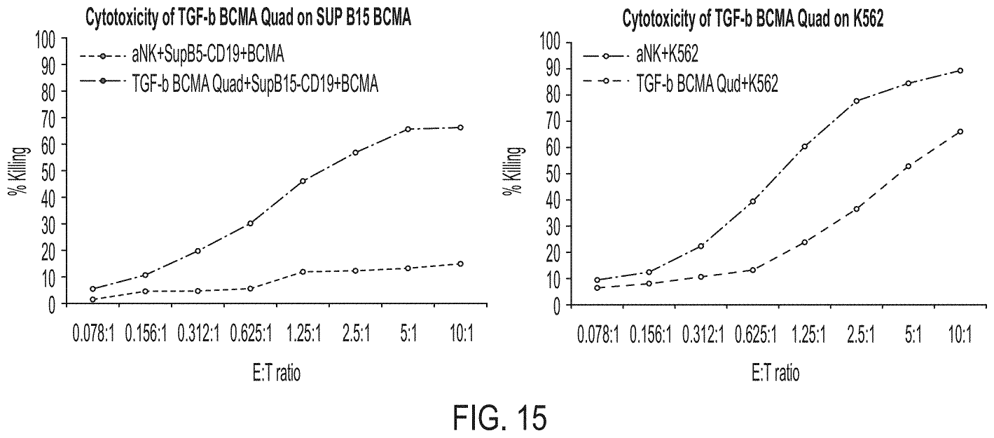

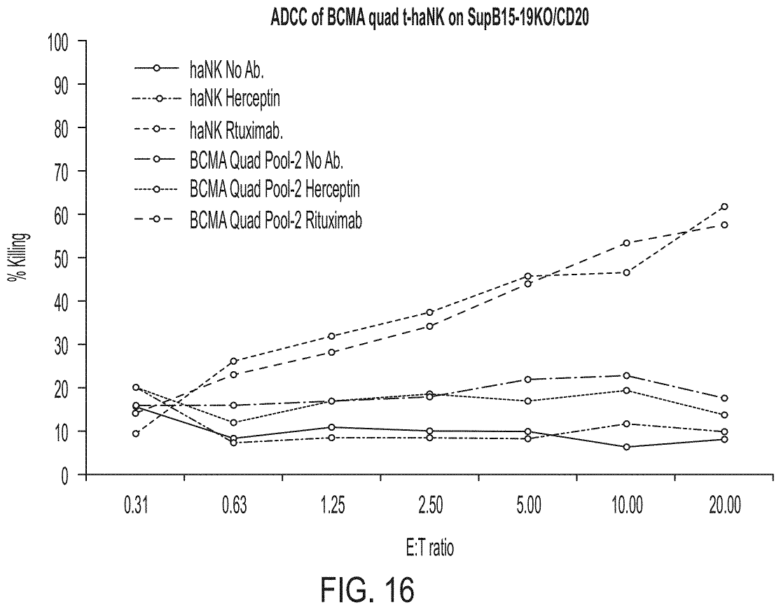

BCMA.CAR t-haNK cells having the quadracistronic vector had strong and target specific CAR-mediated cytotoxicity as can be seen from the results depicted in FIG. 15. ADCC was tested using SUP-B15^{19KO/CD20+} cells and rituximab as target specific antibody and Herceptin as control antibody. Once again, the polyclonal BCMA.CAR t-haNK cells had strong and target specific ADCC approaching about 60% as can be seen from the exemplary results depicted in FIG. 16, with no substantial off-target toxicity.

The BCMA.CAR construct as presented herein may be combined with monoclonal antibodies targeting different antigens expressed on solid tumors, thereby decreasing the risk of relapse due to antigen loss. Furthermore, the quadrcistronic vectors described above confers enhanced homing properties with a chemokine receptor. Furthermore, the BCMA.CAR t-haNK cells described herein may be modified further to express molecules that additionally manipulate/down-regulate the inhibitory factors of the tumor microenvironment. For example, expression of the chemokine receptor CXCR4 on the cell surface of the BCMA.CAR t-haNK cell can direct the cells towards the bone marrow compartment, where the majority of MM cells reside. Secretion of a TGFP trap molecule that can sequester TGFP in the tumor microenvironment or in the bone marrow compartment can counter the immune inhibitory effect of TGFP. It would also counter the stimulating effect of TGFP on osteoclasts, which are responsible for the bone tissue deterioration that is a hallmark of advancing MM.

Of course, it should be recognized that for all nucleic acid sequences provided herein the corresponding encoded proteins are also expressly contemplated herein. Likewise, for all amino acid sequences, corresponding nucleic acids sequences are also contemplated herein (with any codon usage).

All patent applications, publications, references, and sequence accession numbers cited in the present specification are hereby incorporated by reference in their entirety.

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

It is understood that all numerical values described herein (e.g., pH, temperature, time, concentration, amounts, and molecular weight, including ranges) include normal variation in measurements encountered by one of ordinary skill in the art. Thus, numerical values described herein include variation of +/−0.1 to 10%, for example, +/−0.1%, 0.5%, 1%, 2%, 3%, 4%, 5%, 6%, 7%, 8%, 9%, or 10%. It is to be understood, although not always explicitly stated, that all numerical designations may be preceded by the term “about.” Thus, the term about includes variation of +/−0.1 to 10%, for example, +/−0.1%, 0.5%, 1%, 2%, 3%, 4%, 5%, 6%, 7%, 8%, 9%, or 10% of the numerical value. It is also to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein include the end points of the range, and include all values between the end points of the range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this disclosure.

As used herein, “immunotherapy” refers to the use of NK-92 cells, modified or unmodified, naturally occurring or modified NK cell or T-cell, whether alone or in combination, and which are capable of inducing cytotoxicity when contacting a target cell.

As used herein, “natural killer (NK) cells” are cells of the immune system that kill target cells in the absence of a specific antigenic stimulus, and without restriction according to major histocompatibility complex (MHC) class. Target cells may be tumor cells or cells harboring a virus. NK cells are characterized by the presence of CD56 and the absence of CD3 surface markers.

The term “endogenous NK cells” is used to refer to NK cells derived from a donor (or the patient), as distinguished from the NK-92 cell line. Endogenous NK cells are generally heterogeneous populations of cells within which NK cells have been enriched. Endogenous NK cells may be intended for autologous or allogeneic treatment of a patient.

The term “NK-92” refers to natural killer cells derived from the highly potent unique cell line described in Gong et al. (1994), rights to which are owned by NantKwest (hereafter, “NK-92™ cells”). The immortal NK cell line was originally obtained from a patient having non-Hodgkin's lymphoma. Unless indicated otherwise, the term “NK-92™” is intended to refer to the original NK-92 cell lines as well as NK-92 cell lines that have been modified (e.g., by introduction of exogenous genes). NK-92™ cells and exemplary and non-limiting modifications thereof are described in U.S. Pat. Nos. 7,618,817; 8,034,332; 8,313,943; 9,181,322; 9,150,636; and published U.S. Application No. 10/008,955, all of which are incorporated herein by reference in their entireties, and include wild type NK-92™ NK-92™-CD16, NK-92Tm-CD16-γ, NK-92™-CD16-ζ, NK-92™-CD16(F 176V), NK-92™MI, and NK-92™CI. NK-92 cells are known to persons of ordinary skill in the art, to whom such cells are readily available from NantKwest, Inc.

The term “aNK” refers to an unmodified natural killer cells derived from the highly potent unique cell line described in Gong et al. (1994), rights to which are owned by NantKwest (hereafter, “aNK™ cells”). The term “haNK” refers to natural killer cells derived from the highly potent unique cell line described in Gong et al. (1994), rights to which are owned by NantKwest, modified to express CD16 on the cell surface (hereafter, “CD16+NK-92™ cells” or “haNK® cells”). In some embodiments, the CD16+NK-92™ cells comprise a high affinity CD16 receptor on the cell surface. The term “taNK” refers to natural killer cells derived from the highly potent unique cell line described in Gong et al. (1994), rights to which are owned by NantKwest, modified to express a chimeric antigen receptor (hereafter, “CAR-modified NK-92™ cells” or “taNK® cells”). The term “t-haNK” refers to natural killer cells derived from the highly potent unique cell line described in Gong et al. (1994), rights to which are owned by NantkWest, modified to express CD 16 on the cell surface and to express a chimeric antigen receptor (hereafter, “CAR-modified CD16+NK-92™ cells” or “t-haNK™ cells”). In some embodiments, the t-haNK™ cells express a high affinity CD16 receptor on the cell surface.

A “modified NK-92 cell” refers to an NK-92 cell that expresses an exogenous gene or protein, such as an Fc receptor, a CAR, a cytokine (such as IL-2 or IL-15), and/or a suicide gene. In some embodiments, the modified NK-92 cell comprises a vector that encodes for a transgene, such as an Fc receptor, a CAR, a cytokine (such as IL-2 or IL-15), and/or a suicide gene. In one embodiment, the modified NK-92 cell expresses at least one transgenic protein.

As used herein, “non-irradiated NK-92 cells” are NK-92 cells that have not been irradiated. Irradiation renders the cells incapable of growth and proliferation. It is envisioned that the NK-92 cells will be irradiated at the treatment facility or some other point prior to treatment of a patient, since the time between irradiation and infusion should be no longer than four hours in order to preserve optimal activity. Alternatively, NK-92 cells may be prevented from proliferating by another mechanism.

As used herein, “inactivation” of the NK-92 cells renders them incapable of growth. Inactivation may also relate to the death of the NK-92 cells. It is envisioned that the NK-92 cells may be inactivated after they have effectively purged an ex vivo sample of cells related to a pathology in a therapeutic application, or after they have resided within the body of a mammal a sufficient period of time to effectively kill many or all target cells residing within the body. Inactivation may be induced, by way of non-limiting example, by administering an inactivating agent to which the NK-92 cells are sensitive.

As used herein, the terms “cytotoxic” and “cytolytic,” when used to describe the activity of effector cells such as NK-92 cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK-92 cells is due to cytolysis.

The term “kill” with respect to a cell/cell population is directed to include any type of manipulation that will lead to the death of that cell/cell population.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FCγR) bind to the IgG class of antibodies. FCγRIII-A is a low affinity Fc receptor bind to IgG antibodies and activate ADCC. FCγRIII-A are typically found on NK cells. NK-92 cells do not express FCγRIII-A. Fc-epsilon receptors (FcεR) bind to the Fc region of IgE antibodies.

The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. A CAR-expressing NK-92 cell is targeted to cells expressing certain antigens on the cell surface, based on the specificity of the scFv domain. The scFv domain can be engineered to recognize any antigen, including tumor-specific antigens and virus-specific antigens. For example, CD19CAR recognizes CD19, a cell surface marker expressed by some cancers.

The term “tumor-specific antigen” as used herein refers to antigens that are present on a cancer or neoplastic cell but not detectable on a normal cell derived from the same tissue or lineage as the cancer cell. Tumor-specific antigens, as used herein, also refers to tumor-associated antigens, that is, antigens that are expressed at a higher level on a cancer cell as compared to a normal cell derived from the same tissue or lineage as the cancer cell.

The term “virus-specific antigen” as used herein refers to antigens that are present on a virus-infected cell but not detectable on a normal cell derived from the same tissue or lineage as the virus-infected cell. In one embodiment, a virus-specific antigen is a viral protein expressed on the surface of an infected cell.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, “percent identity” refers to sequence identity between two peptides or between two nucleic acid molecules. Percent identity can be determined by comparing a position in each sequence which may be aligned for purposes of comparison. When a position in the compared sequence is occupied by the same base or amino acid, then the molecules are identical at that position. Homologous nucleotide sequences include those sequences coding for naturally occurring allelic variants and mutations of the nucleotide sequences set forth herein. Homologous nucleotide sequences include nucleotide sequences encoding for a protein of a mammalian species other than humans. Homologous amino acid sequences include those amino acid sequences which contain conservative amino acid substitutions and which polypeptides have the same binding and/or activity. In some embodiments, a homologous amino acid sequence has no more than 15, nor more than 10, nor more than 5 or no more than 3 conservative amino acid substitutions. In some embodiments, a nucleotide or amino acid sequence has at least 60%, at least 65%, at least 70%, at least 80%, or at least 85% or greater percent identity to a sequence described herein. In some embodiments, a nucleotide or amino acid sequence has at least 90%, 91%, 92%, 93%, 94%, 95%, 96%, 97%, 98% or 99% identity to a sequence described herein. Percent identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). Algorithms suitable for determining percent sequence identity include the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (see the internet at ncbi.nlm.nih.gov). The BLAST algorithm parameters W, T, and X determine the sensitivity and speed of the alignment. The BLASTN program (for nucleotide sequences) uses as defaults a wordlength (W) of 11, an expectation (E) or 10, M=5, N=-4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4.

In some embodiments, a nucleic acid sequence is codon optimized for expression in a particular species, for example, a mouse sequence can be codon optimized for expression in humans (expression of the protein encoded by the codon-optimized nucleic acid sequence). Thus, in some embodiments, a codon-optimized nucleic acid sequence has at least 60%, at least 65%, at least 70%, at least 80%, or at least 85% or greater percent identity to a nucleic acid sequence described herein. In some embodiments, a codon-optimized nucleic acid sequence acid sequence has at least 90%, 91%, 92%, 93%, 94%, 95%, 96%, 97%, 98% or 99% identity to a sequence described herein.

The term “express” refers to the production of a gene product (e.g., a protein). The term “transient” when referring to expression means a polynucleotide is not incorporated into the genome of the cell. The term “stable” when referring to expression means a polynucleotide is incorporated into the genome of the cell, or a positive selection marker (i.e., an exogenous gene expressed by the cell that confers a benefit under certain growth conditions) is utilized to maintain expression of the transgene.

The term “cytokine” or “cytokines” refers to the general class of biological molecules which affect cells of the immune system. Exemplary cytokines include but are not limited to interferons and interleukins (IL) —in particular IL-2, IL-12, IL-15, IL-18 and IL-21. In preferred embodiments, the cytokine is IL-2.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited or no ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA. In one embodiment, the vector is a viral vector, e.g. adenovirus. Viral vectors are well known in the art.

As used herein, the term “targeted,” when referring to protein expression, is intended to include, but is not limited to, directing proteins or polypeptides to appropriate destinations in the cell or outside of it. The targeting is typically achieved through signal peptides or targeting peptides, which are a stretch of amino acid residues in a polypeptide chain. These signal peptides can be located anywhere within a polypeptide sequence, but are often located on the N-terminus. Polypeptides can also be engineered to have a signal peptide on the C-terminus. Signal peptides can direct a polypeptide for extracellular section, location to plasma membrane, golgi, endosomes, endoplasmic reticulum, and other cellular compartments. For example, polypeptides with a particular amino acid sequence on their C-terminus (e.g., KDEL) are retained in the ER lumen or transported back the ER lumen.

As used herein, the term “target,” when referring to targeting of a tumor, refers to the ability of NK-92 cells to recognize and kill a tumor cell (i.e., target cell). The term “targeted” in this context refers, for example, to the ability of a CAR expressed by the NK-92 cell to recognize and bind to a cell surface antigen expressed by the tumor.

As used herein, the term “transfect” refers to the insertion of nucleic acid into a cell. Transfection may be performed using any means that allows the nucleic acid to enter the cell. DNA and/or mRNA may be transfected into a cell. Preferably, a transfected cell expresses the gene product (i.e., protein) encoded by the nucleic acid.

The term “suicide gene” refers to a transgene that allows for the negative selection of cells expressing that transgene. A suicide gene is used as a safety system, allowing the cells expressing the gene to be killed by introduction of a selective agent. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene (see also, for example, Yazawa K, Fisher W E, Brunicardi F C: Current progress in suicide gene therapy for cancer. World J. Surg. 2002 Jul.; 26(7):783-9). In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir.

1. A genetically modified NK cell expressing
(i) a membrane bound recombinant chimeric antigen receptor (CAR) that comprises in a single polypeptide chain an extracellular binding domain, a hinge domain, a transmembrane domain, and a signaling domain, wherein the extracellular binding domain specifically binds to a BCMA receptor;
(ii) a recombinant CD16;
(iii) an autocrine growth stimulating cytokine; and
(iv) optionally one of IL-12, a TGF-beta trap, or a homing receptor.

2-7. (canceled)

8. The genetically modified NK cell of claim 1 wherein the IL-12 is a single chain IL-12 heterodimer.

9. The genetically modified NK cell of claim 1 wherein the TGF-beta trap comprises a single chain dimer of the TGF-beta Receptor II ectodomain.

10-12. (canceled)

13. A genetically modified NK cell, comprising:
a recombinant nucleic acid encoding
(i) a membrane bound recombinant chimeric antigen receptor (CAR) that comprises in a single polypeptide chain an extracellular binding domain, a hinge domain, a transmembrane domain, and a signaling domain, wherein the extracellular binding domain specifically binds to a BCMA receptor;
(ii) a recombinant CD16;
(iii) an autocrine growth stimulating cytokine; and
(iv) optionally one of IL-12, a TGF-beta trap, or a homing receptor.

14. The genetically modified NK cell of claim 13 wherein the recombinant nucleic acid is a polycistronic RNA.

15. The genetically modified NK cell of claim 13 wherein the NK cell is an NK-92 cell.

16. The genetically modified NK cell of claim 13 wherein the extracellular binding domain comprises a scFv.

17. The genetically modified NK cell of claim 13 wherein the signaling domain comprises a FcεRIγ signaling domain or a CD3ζ signaling domain.

18. The genetically modified NK cell of claim 13 wherein the recombinant CD16 is a CD1 6^{158V} mutant.

19. The genetically modified NK cell of claim 13 wherein the autocrine growth stimulating cytokine is IL-2 or IL-15.

20. The genetically modified NK cell of claim 19 wherein the autocrine IL-2 or IL-15 further comprises an endoplasmic retention sequence.

21. The genetically modified NK cell of claim 13 wherein the IL-12 is a single chain IL-12 heterodimer.

22. The genetically modified NK cell of claim 13 wherein the TGF-beta trap comprises a single chain dimer of the TGF-beta Receptor II ectodomain.

23. The genetically modified NK cell of claim 22 wherein the TGF-beta trap is a secreted form of a single chain dimer of the TGF-beta Receptor II ectodomain.

24. The genetically modified NK cell of claim 13 wherein the homing receptor is a cell adhesion molecule, a selectin, an integrin, a C—C chemokine receptor, or C—X—C chemokine receptor.

25. The genetically modified NK cell of claim 24 wherein the homing receptor is selected from the group consisting of CCR1, CCR2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1, CCXCKR, D6, DARC, and a receptor for CXCL14.

26. A method of treating cancer in a patient in need thereof, comprising administering to the patient a therapeutically effective amount of any one of the genetically modified NK cells of claim 1 or claim 13, thereby treating the cancer.

27. The method of claim 26 further comprising a step of administering at least one additional therapeutic entity selected from the group consisting of a viral cancer vaccine, a bacterial cancer vaccine, a yeast cancer vaccine, N-803, an antibody, a stem cell transplant, and a tumor targeted cytokine.

28. The method of claim 26, wherein the cancer is multiple myeloma or Hodgkin lymphoma.

29. The method of claim 26, wherein about 1×10⁸ to about 1×10¹¹ cells per m² of body surface area of the patient are administered to the patient.

30. (canceled)


SEQUENCE LISTING (42 sequences)

SEQ ID NO: 1 - protein, 41 aa, Artificial Sequence
  FC-epsilon-RI-gamma cytoplasmic domain
LKIQVRKAAITSYEKSDGVYTGLSTRNQETYETLKHEKPPQ

SEQ ID NO: 2 - DNA, 123 bp, Artificial Sequence
  FC-epsilon-RI-gamma cytoplasmic domain
ctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 3 - protein, 54 aa, Artificial Sequence
  CD8 hinge
KPTTTPAPRPPTPAPTIASQPLSLRPEACRPAAGGAVHTRGLDFACFWVLVVVG

SEQ ID NO: 4 - protein, 20 aa, Artificial Sequence
  CD28 transmembrane domain
GVLACYSLLVTVAFIIFWVR

SEQ ID NO: 5 - protein, 41 aa, Artificial Sequence
  CD28 signaling domain
RSKRSRLLHSDYMNMTPRRPGPTRKHYQPYAPPRDFAAYRS

SEQ ID NO: 6 - protein, 42 aa, Artificial Sequence
  4-1BB signaling domain
KRGRKKLLYIFKQPFMRPVQTTQEEDGCSCRFPEEEEGGCEL

SEQ ID NO: 7 - protein, 113 aa, Artificial Sequence
  CD3 zeta signaling domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - DNA, 339 bp, Artificial Sequence
  CD3 zeta
gtgaagtttagcagatctgccgacgcccctgcctaccagcagggacagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagataa

SEQ ID NO: 9 - protein, 153 aa, Artificial Sequence
  Human IL2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 160 aa, Artificial Sequence
  Human er-IL2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

SEQ ID NO: 11 - protein, 254 aa, Artificial Sequence
  FCgamma/CD16
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 12 - DNA, 765 bp, Artificial Sequence
  FCgamma/CD16 nt
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 13 - DNA, 1125 bp, Artificial Sequence
  CCR7
gccaccatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccatag

SEQ ID NO: 14 - DNA, 297 bp, Artificial Sequence
  CCL19
atggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaa

SEQ ID NO: 15 - DNA, 405 bp, Artificial Sequence
  CCL21
atggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggccatag

SEQ ID NO: 16 - DNA, 1083 bp, Artificial Sequence
  CXCR2 nt
atggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaa

SEQ ID NO: 17 - protein, 360 aa, Artificial Sequence
  CXCR2 aa
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 18 - DNA, 336 bp, Artificial Sequence
  CXCL4 nt
atgtccctgctcccacgccgcgcccctccggtcagcatgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatag

SEQ ID NO: 19 - protein, 111 aa, Artificial Sequence
  CXCL4 aa
MSLLPRRAPPVSMRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 20 - DNA, 1119 bp, Artificial Sequence
  CD62L nt
atgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaa

SEQ ID NO: 21 - protein, 372 aa, Artificial Sequence [sequence omitted]

SEQ ID NO: 22 - DNA, 300 bp, Artificial Sequence
  IL8 nt
atgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataa

SEQ ID NO: 23 - protein, 99 aa, Artificial Sequence
  IL8 aa
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 24 - DNA, 324 bp, Artificial Sequence
  CXCL1 nt
atggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactga

SEQ ID NO: 25 - protein, 107 aa, Artificial Sequence
  CXCL1 aa
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 26 - DNA, 762 bp, Artificial Sequence
  IL-12 p35 nt
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 27 - protein, 253 aa, Artificial Sequence
  IL-12 p35 aa
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 28 - DNA, 987 bp, Artificial Sequence
  IL-12 p40 nt
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 29 - protein, 328 aa, Artificial Sequence
  IL-12 p40 aa
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 30 - protein, 536 aa, Artificial Sequence
  IL-12 single chain p40_p35 aa
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - DNA, 1611 bp, Artificial Sequence
  IL-12 single chain p40_p35 nt
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 32 - DNA, 477 bp, Artificial Sequence
  TGF-beta receptor II extracellular domain nt
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 33 - protein, 159 aa, Artificial Sequence
  TGF-beta receptor II extracellular domain aa
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 34 - DNA, 933 bp, Artificial Sequence
  TGF-beta trap nt
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggaggtggaggaagtggaggaggtggaagtggtggtggaggtagtacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 35 - protein, 311 aa, Artificial Sequence
  TGF-beta trap aa
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 36 - DNA, 510 bp, Artificial Sequence
  erIL-15 nt
atgcgcatcagcaagccccacctgcgcagcatcagcatccagtgctacctgtgcctgctgctgaacagccacttcctgaccgaggccggcatccacgtgttcatcctgggctgcttcagcgccggcctgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggctccgagaaggacgagctgtaa

SEQ ID NO: 37 - protein, 169 aa, Artificial Sequence
  erIL-15 aa
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSEKDEL

SEQ ID NO: 38 - DNA, 1194 bp, Artificial Sequence
  BCMA CAR
atggactggatctggcggattctgtttctcgtgggagctgccacaggcgctcattctgctcagcctgccgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaggcggcggaggaagcggaggcggaggatctgggggcggaggctctgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaagcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 39 - DNA, 786 bp, Artificial Sequence
  BCMA scFv DNA sequence VL/VH format
atggactggatctggcggattctgtttctcgtgggagctgccacaggcgctcattctgctcagcctgccgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaaggcggcggaggaagcggaggcggaggatctgggggcggaggctctgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctca

SEQ ID NO: 40 - DNA, 786 bp, Artificial Sequence
  BCMA scFv DNA sequence VH/VL format
atggactggatctggcggattctgtttctcgtgggagctgccacaggcgctcattctgctcagcctgccgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaggcggcggaggaagcggaggcggaggatctgggggcggaggctctgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaa

SEQ ID NO: 41 - protein, 262 aa, Artificial Sequence
  BCMA scFv Protein sequence VL/VH format
MDWIWRILFLVGAATGAHSAQPADIVMTQSPATLSVTPGDRVSLSCRASQSISDYLHWYQQKSHESPRLLIKYASQSISGIPSRFSGSGSGSDFTLSINSVEPEDVGVYYCQNGHSFPPTFGGGTKLEIKGGGGSGGGGSGGGGSVQLQQSGPELVKPGASVKMSCKASGYTFTSYVMHWVKQKPGQGLEWIGYIIPYNDATKYNEKFKGKATLTSDKSSSTAYMELSSLTSEDSAVYYCARYNYDGYFDVWGAGTTVTVSS

SEQ ID NO: 42 - protein, 262 aa, Artificial Sequence
  BCMA scFv Protein sequence VH/VL format
MDWIWRILFLVGAATGAHSAQPAVQLQQSGPELVKPGASVKMSCKASGYTFTSYVMHWVKQKPGQGLEWIGYIIPYNDATKYNEKFKGKATLTSDKSSSTAYMELSSLTSEDSAVYYCARYNYDGYFDVWGAGTTVTVSSGGGGSGGGGSGGGGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPPTFGGGTKLEIK